SEQUENCE LISTING (55 sequences)

SEQ ID NO: 1 - DNA, 22 bp, Synthetic construct
  source 1..22: mol_type = other DNA
tgagcacaaatcctaaacctca

SEQ ID NO: 2 - DNA, 20 bp, Synthetic construct
  source 1..20: mol_type = other DNA
ttcctcacaggggagtgatt

SEQ ID NO: 3 - DNA, 26 bp, Synthetic construct
  source 1..26: mol_type = other DNA
caaatcctaaacctcaaagaaaaacc

SEQ ID NO: 4 - DNA, 22 bp, Synthetic construct
  source 1..22: mol_type = other DNA
ctcacaggggagtgattcatgg

SEQ ID NO: 5 - DNA, 23 bp, Synthetic construct
  source 1..23: mol_type = other DNA
ccagtctcgccaaacatggctgt

SEQ ID NO: 6 - DNA, 29 bp, Synthetic construct
  source 1..29: mol_type = other DNA
caccatgtagctgctactggtattcttca

SEQ ID NO: 7 - DNA, 26 bp, Synthetic construct
  source 1..26: mol_type = other DNA
gacgcacatcgatatggttgtgatgt

SEQ ID NO: 8 - DNA, 22 bp, Synthetic construct
  source 1..22: mol_type = other DNA
ggaacggtgatgcaggacaaca

SEQ ID NO: 9 - DNA, 22 bp, Synthetic construct
  source 1..22: mol_type = other DNA
ccctatatgggaatgagggact

SEQ ID NO: 10 - DNA, 23 bp, Synthetic construct
  source 1..23: mol_type = other DNA
cgttagggtgtcgatgactttac

SEQ ID NO: 11 - DNA, 22 bp, Synthetic construct
  source 1..22: mol_type = other DNA
cgagatgtgatgaaggagatgg

SEQ ID NO: 12 - DNA, 22 bp, Synthetic construct
  source 1..22: mol_type = other DNA
ttgatgtaatccagcaggtcag

SEQ ID NO: 13 - DNA, 42 bp, Synthetic construct
  source 1..42: mol_type = other DNA
cttggtaccgagctcggatccactagtccagtgtggtggaat

SEQ ID NO: 14 - DNA, 33 bp, Synthetic construct
  source 1..33: mol_type = other DNA
ttattaattaagtcgactgcagaattcagatcc

SEQ ID NO: 15 - DNA, 35 bp, Synthetic construct
  source 1..35: mol_type = other DNA
gtcctgcagggtatggtgagcaagggcgaggagct

SEQ ID NO: 16 - DNA, 40 bp, Synthetic construct
  source 1..40: mol_type = other DNA
aacgggccctctagactcgagcggccgcctgaggtgccac

SEQ ID NO: 17 - DNA, 38 bp, Synthetic construct
  source 1..38: mol_type = other DNA
gcagtcgacttaattaataataggggcgacactccgcc

SEQ ID NO: 18 - DNA, 39 bp, Synthetic construct
  source 1..39: mol_type = other DNA
ctcaccataccctgcaggacgtcttctgggcgacggttg

SEQ ID NO: 19 - RNA, 23 nt, Synthetic construct
  source 1..23: mol_type = other RNA
cgccaccttgctggccctgtttt

SEQ ID NO: 20 - RNA, 23 nt, Synthetic construct
  source 1..23: mol_type = other RNA
gccaccttgctggccctgttgtt

SEQ ID NO: 21 - RNA, 21 nt, Synthetic construct
  source 1..21: mol_type = other RNA
gatcatacgtgcgatcagatt

SEQ ID NO: 22 - RNA, 21 nt, Synthetic construct
  source 1..21: mol_type = other RNA
tctgatcgcacgtatgatctt

SEQ ID NO: 23 - DNA, 31 bp, Synthetic construct
  source 1..31: mol_type = other DNA
aaaagcaggctactcgaattctaatacgact

SEQ ID NO: 24 - DNA, 41 bp, Synthetic construct
  source 1..41: mol_type = other DNA
aacagccgccagccgctcacgggagtgattcctggcggagt

SEQ ID NO: 25 - DNA, 35 bp, Synthetic construct
  source 1..35: mol_type = other DNA
agttagctaatcttcttgaacagccgccagccgct

SEQ ID NO: 26 - DNA, 39 bp, Synthetic construct
  source 1..39: mol_type = other DNA
ttcaagaagattagctaactgtgaggaactactgtcttc

SEQ ID NO: 27 - DNA, 20 bp, Synthetic construct
  source 1..20: mol_type = other DNA
cacgcgatgccatcgcggcc

SEQ ID NO: 28 - DNA, 31 bp, Synthetic construct
  source 1..31: mol_type = other DNA
cgctcacgggagtgattcatggcggagtgtc

SEQ ID NO: 29 - DNA, 26 bp, Synthetic construct
  source 1..26: mol_type = other DNA
gccgccagccgctcacgggagtgatt

SEQ ID NO: 30 - DNA, 46 bp, Synthetic construct
  source 1..46: mol_type = other DNA
atcgaaattaatacgactcactataggacctgcccctaataggggc

SEQ ID NO: 31 - DNA, 33 bp, Synthetic construct
  source 1..33: mol_type = other DNA
gccctctagactcgaacccagtcttcccggcaa

SEQ ID NO: 32 - DNA, 23 bp, Synthetic construct
  source 1..23: mol_type = other DNA
ttaacgtcttctgggcgacggtt

SEQ ID NO: 33 - RNA, 23 nt, Synthetic construct
  source 1..23: mol_type = other RNA
tggagtgtgacaatggtgtttgt

SEQ ID NO: 34 - RNA, 45 nt, Synthetic construct
  source 1..45: mol_type = other RNA
acctgcccctaataggggcgacactccgccatgaatcactcccct

SEQ ID NO: 35 - protein, 11 aa, Synthetic construct
VSGWRLFKKIS

SEQ ID NO: 36 - DNA, 81 bp, Synthetic construct
  source 1..81: mol_type = other DNA
acctgcccctaataggggcgacactccgccaggaatcactcccgtgagcggctggcggctgttcaagaagattagctaact

SEQ ID NO: 37 - DNA, 81 bp, Synthetic construct
  source 1..81: mol_type = other DNA
acctgcccctaataggggcgacactccgccatgaatcactcccgtgagcggctggcggctgttcaagaagattagctaact

SEQ ID NO: 38 - DNA, 279 bp, Synthetic construct
  source 1..279: mol_type = other DNA
tgctggccctgttgtcctgcatcaccgttccggtctctgctgcccaggtgaaaaataccagtagcagctacatggtgaccaatgactgctccaatgacagcatcacttggcagctcgaggctgcggttctccacgtccccgggtgcgtcccgtgcgagagagtggggaatacgtcacggtgttgggtgccagtctcgccaaacatggctgtgcggcagctcggtgccctcacgcagggtctgcggacgcacatcgatatggttgtgatgtccgccacct

SEQ ID NO: 39 - DNA, 35 bp, Synthetic construct
  source 1..35: mol_type = other DNA
tctgcagtcgacttacatgaatcactcccctgtga

SEQ ID NO: 40 - DNA, 40 bp, Synthetic construct
  source 1..40: mol_type = other DNA
ttgctcaccatacccgtttttctttgaggtttaggatttg

SEQ ID NO: 41 - DNA, 40 bp, Synthetic construct
  source 1..40: mol_type = other DNA
tctgcagtcgacttaattaaaggggcgacactccgccatg

SEQ ID NO: 42 - DNA, 41 bp, Synthetic construct
  source 1..41: mol_type = other DNA
ttgctcaccatacccagaggtttaggatttgtgctcatggt

SEQ ID NO: 43 - DNA, 35 bp, Synthetic construct
  source 1..35: mol_type = other DNA
tctgcagtcgacttaccgacatgaatcactcccct

SEQ ID NO: 44 - DNA, 39 bp, Synthetic construct
  source 1..39: mol_type = other DNA
ttgctcaccataccctttctttgaggtttaggatttgtg

SEQ ID NO: 45 - DNA, 33 bp, Synthetic construct
  source 1..33: mol_type = other DNA
tctgcagtcgacttattgctggccctgttgtcc

SEQ ID NO: 46 - DNA, 34 bp, Synthetic construct
  source 1..34: mol_type = other DNA
ttgctcaccatacccggtggcggacatcacaacc

SEQ ID NO: 47 - DNA, 18 bp, Synthetic construct
  source 1..18: mol_type = other DNA
gccaccttgctggccctg

SEQ ID NO: 48 - DNA, 18 bp, Synthetic construct
  source 1..18: mol_type = other DNA
cgccaccttgctggccct

SEQ ID NO: 49 - DNA, 17 bp, Synthetic construct
  source 1..17: mol_type = other DNA
gtccgccaccttcatct

SEQ ID NO: 50 - DNA, 17 bp, Synthetic construct
  source 1..17: mol_type = other DNA
ccgccaccttctatctt

SEQ ID NO: 51 - RNA, 45 nt, Synthetic construct
  source 1..45: mol_type = other RNA
acctgcccctaataggggcgacactccgccaggaatcactcccct

SEQ ID NO: 52 - DNA, 18 bp, Synthetic construct
  source 1..18: mol_type = other DNA
gaagacgttaataggggc

SEQ ID NO: 53 - DNA, 18 bp, Synthetic construct
  source 1..18: mol_type = other DNA
gaagacgttaataggggc

SEQ ID NO: 54 - DNA, 18 bp, Synthetic construct
  source 1..18: mol_type = other DNA
cctaaacctcaggggcga

SEQ ID NO: 55 - DNA, 17 bp, Synthetic construct
  source 1..17: mol_type = other DNA
ctaaacctcaggggcga

(19) United States
(12) Patent Application Publication
(10) Pub. No.: US 2025/0034573 A1
(43) Pub. Date: Jan. 30, 2025

(54) CIRCULAR RNA DERIVED FROM RNA VIRUSES AND RELATED COMPOSITIONS AND METHODS

(71) Applicants: CZ Biohub SF, LLC, San Francisco, CA (US); The Board of Trustees of the Leland Stanford Junior University, Stanford, CA (US)

(72) Inventors: Peter Sarnow, Stanford, CA (US); Qian Cao, Stanford, CA (US)

(21) Appl. No.: 18/716,201

(22) PCT Filed: Jan. 4, 2023
(86) PCT No.: PCT/US2023/060109

§ 371 Date: Jun. 4, 2024

Related U.S. Application Data
(60) Provisional application No. 63/296,360, filed on Jan. 4, 2022.

(51) Int. Cl. C12N 15/113; C12Q 1/70

(52) U.S. Cl. CPC C12N 15/1131; C12Q 1/707; C12N 2310/14; C12Q 2600/156

(57) ABSTRACT

Provided herein are methods and compositions for reducing the proviral effect of a virus-derived circular RNA on a target cell through the use of an agent that blocks a proviral function of the virus-derived circular RNA. Also, provided herein are methods of treating a cytoplasmic or nuclear RNA viral infection in a subject and compositions to block a proviral effect of a virus-derived circular RNA or compositions that promote an antiviral effect of the virus-derived circular RNA.

### CROSS-REFERENCE TO RELATED APPLICATION

This application claims priority to Provisional Patent Application No. 63/296,360, filed Jan. 4, 2022, which is incorporated by reference herein in its entirety.

### STATEMENT OF FEDERALLY SPONSORED RESEARCH AND DEVELOPMENT

This invention was made with Government support under contract AI069000 awarded by the National Institutes of Health. The Government has certain rights in the invention.

### BACKGROUND

Effective treatment of viral infections continues to be a challenge. Direct-acting antivirals (DAA), such as anti-polymerase and anti-proteinase compounds, have been shown to be effective against RNA viral infections in many patients. These treatments, however, are costly and unavailable to most people worldwide, and it is still unknown whether every patient population will respond to the current DAA treatments. Thus, it is important to continue to search for new compounds that target both viral and host susceptibility factors to combat RNA virus infections.

### SUMMARY

As shown herein, genomic and antigenomic RNA in RNA viruses can be processed during viral infection to yield hundreds of different virus-derived circular RNAs. The virus-derived circular RNAs can be generated from all parts of the viral RNA genome and can contain internal ribosome entry site (IRES) sequences. Importantly, virus-derived circular RNAs can have pro-viral functions and can be translated to yield novel proteins. Thus, virus-derived circular RNAs present a novel class of viral RNA species with novel functions in infected and uninfected bystander cells.

The present disclosure provides a method of reducing a proviral effect of a virus-derived circular RNA on a target cell. The method comprises first identifying a virus-derived circular RNA having a proviral effect, wherein the virus-derived circular RNA is derived from a genome of a cytoplasmic RNA virus (e.g., a flavivirus, a coronavirus, a reovirus, a picornavirus, or a togavirus) or a nuclear RNA virus (e.g., an influenza or a retrovirus). The method further comprises contacting the target cell with an agent that blocks a proviral function of the virus-derived circular RNA. The agent can be, for example, a siRNA, an shRNA, an RNA-targeting CRISPR-Cas system, or a small molecule, and the target cell can be an infected cell or a bystander cell.

The present disclosure also provides a method of promoting an innate immune response in a subject by administering to the subject a virus-derived circular RNA of a cytoplasmic or nuclear RNA virus, wherein the selected virus-derived circular RNA promotes in the subject an innate immune response to the RNA virus. Also provided is a method of promoting an innate immune response in a subject by administering to the subject an agent that blocks a function of a virus-derived circular RNA of a RNA virus, wherein the selected virus-derived circular RNA reduces in the subject an innate immune response to the RNA virus.

A method of treating an RNA viral infection in a subject is also provided. The method comprises detecting in a biological sample from the subject a virus-derived circular RNA derived from the genome of an RNA virus or by detecting a polypeptide translated from a virus-derived circular RNA and administering to the subject a therapeutically effective amount of an antiviral agent.

The present disclosure also provides a vector encoding an siRNA, an shRNA, a RNA-targeting CRISPR-Cas system, or a small molecule that targets a virus-derived circular RNA, wherein the virus-derived circular RNA is derived from a genome of an RNA virus.

Also described herein are nanoparticles comprising an agent that blocks a function of a virus-derived circular RNA and a polymer layer encapsulating or containing the agent. Optionally, the nanoparticles comprise a vector encoding an siRNA, an shRNA, or a RNA-targeting CRISPR-Cas system that targets a virus-derived circular RNA derived from a genome of an RNA virus.

The present disclosure provides a method of modulating an effect of a virus-derived circular RNA on one or more uninfected bystander cells. The method comprises contacting the one or more uninfected bystander cells with a therapeutically effective amount of an agent that blocks a proviral effect of the virus-derived circular RNA on the one or more uninfected bystander cells or an agent that promotes an antiviral effect of the virus-derived circular RNA in the one or more uninfected bystander cells.

### BRIEF DESCRIPTION OF THE DRAWINGS

The present disclosure may be better understood by reference to the following non-limiting figures.

FIG. 1A-D show the landscape of HCV-derived circRNAs (vcircRNA) predicted by RNA-seq analysis. FIG. 1A is a schematic showing formation of a circRNA from the linear HCV RNA genome. The curved arrow in black represents a vcircRNA junction-spanning read. In the alignment, the four nucleotide positions in discontinuous sequences are denoted as 5′ (labelled a) and 3′ (labelled b) breakpoints. The start (labelled c) and the end positions (labelled d) of reads on the viral genome are indicated. FIG. 1B shows the predicted distribution of vcircRNA junctions on the entire HCV genome. Each junction read is indicated as an arch connecting its 5′ break and 3′ break point. FIG. 1C is a list of the top 20 predicted vcircRNA junctions. FIG. 1D shows the length distribution of the vcircRNA predicted from junction-spanning reads.

FIG. 2A is a schematic view of vcircRNAs and their linear counterparts (left panel) during reverse transcription (middle panel) and subsequent PCR (right panel) using divergent primers that flank the junction sites (JS) of predicted vcircRNAs for identification of HCV vcircRNAs in infected Huh7 cells by rolling circle amplification. RT-PCR amplification of one or more of the vcircRNA sequence copies generated by rolling circle amplification are denoted as a, b, c, and d (right panel). FIG. 2B-C shows agarose gel electrophoreses of RT-PCR products. Two groups of products representing multiple copies of vcircRNAs were detected using specific primers: the most abundant vcircRNA˜870/˜1151 and IRES-containing vcircRNA˜10/˜400. FIG. 2D shows resistance to RNase R treatment of vcircRNA ˜870/˜1151. Lanes 2 and 3 are duplicates of untreated samples. Lanes 5 and 6 are duplicates of RNase R treated samples. FIG. 2E shows representative sequencing results of vcircRNAs that contain at least one full copy of circRNA sequence and two flanking junction sites. Junction sites are indicated by triangles. Sequencing was performed on vcircRNA cDNAs cloned into TOPO plasmids. From top to bottom the sequences comprises SEQ ID NO:47 (left) and SEQ ID NO:48 (right); SEQ ID NO: 49 (left) and 50 (right); SEQ ID NO:52 (left) and SEQ ID NO:53 (right); and SEQ ID NO: 54 (left) and SEQ ID NO: 55 (right). FIG. 2F shows detection of vcircRNAs from in vitro-transcribed HCV RNA and RNAs isolated from HCV-infected cells. RT-PCR was performed using primers that flank the junctions of predictive IRES-containing vcircRNAs. cDNAs were displayed in a 1% agarose gel. Results from two independent experiments are shown.

FIG. 3A-B show overexpression plasmids containing vcircRNA IRESs and quantification of vcircRNA abundance during viral infection. FIG. 3A shows schematic views of a split GFP plasmid that contains the IRES sequence to generate circular GFP. FIG. 3B is a graph showing the results of droplet digital PCR used to quantify the IRES-containing vcircRNAs with a 5′ breakpoint in proximity to nucleotide 10 and 3′ breakpoint around nucleotide 400. Total RNAs were extracted from mock-infected cells and JFH-1-infected cells (at 3 dpi). Full-length IVT RNAs were used as a negative control for vcircRNAs generated from virally infected cells.

FIG. 4A provides fluorescence micrographs showing translation of the HCV IRES in circular RNAs expressed from a polymerase II promoter-containing split-GFP plasmid. Huh7 cells were transfected individually with plasmids expressing the IRES from encephalomyocarditis virus (EMCV) or various sequence elements derived from distinct vcircRNAs. At 48 hours post transfection, GFP was examined by fluorescent microscopy. The scale bar represents 500 μm. FIG. 4B provides schematics of three infectious clones of HCV that contain a HiBiT tag of 11 amino acids inserted after microRNA 122 binding sites in the 5′UTR to allow microRNA 122 binding. In HCV-10/405U-Hibit, an in-frame stop codon (UGA, the U at position 405 is highlighted in gray) in vcirc 10/405 ORF precedes the HiBiT tag and prevents its translation. However, HCV-10/405G-Hibit has a G mutation at position 405 to eliminate the upstream UGA codon and allow the expression of the HiBiT tag in vcirc 10/405 ORF, should translation have initiated from the IRES in this context. In the HCV-25/1277-Hibit mutant, HiBiT is inserted in-frame with the vcirc25/1277 ORF that has a stop codon at 78 nt in the 5′ UTR. FIG. 4C is a graph showing HiBiT expression from infectious viral genomes described above. Full-length viral RNAs were generated and transfected into Huh7 cells. HiBiT expression from wildtype HCV or mutants were measured as nanoluciferase activities at indicated time points. FIG. 4D shows the effects of IRES-containing vcircRNAs on viral expression of HiBIT. Huh7 cells were transfected with control siRNA (siRNA-Ctrl) or pooled siRNAs (pooled siRNA-circIRES) targeting the junction sites of three IRES-containing vcircRNAs (10/405, 14/362, 30/372) at a final concentration of 100 nM. At 1 day post transfection, cells were transfected with full-length viral RNA of HCV-10/405G-Hibit or HCV-25/1277-Hibit. HiBIT expression was further analyzed at 1 day after viral genome transfection. FIG. 4E shows the effects of IRES-containing vcircRNAs on HCV infection. Huh7 cells were transfected with siRNAs as described above. At 1 day post transfection, cells were further infected with HCV JFH-1 strain at an moi of 0.5. At 2 days post infection (dpi), viral RNA abundances were quantified by qRT-PCR using primers that detect the viral 5′UTR and core coding sequences. *p<0.05.

FIG. 5 is a graph showing translation of the HiBiT tagged linear HCV RNAs. Linear RNA fragments containing HiBiT were generated by in vitro transcription of PCR products amplified from 5′ UTRs of wildtype or HiBiT-inserted HCV JFH-1 mutants. Cells were transfected with linear RNAs of different lengths. HiBiT expression was subsequently quantified by the Nano-Glo HiBiT assay. The legend corresponds to columns from left to right in the graph.

FIG. 6 shows construction of full-length HCV JFH-1 infectious clones carrying a Nanoluciferase insertion and the effects of IRES-containing vcircRNA knockdown on viral RNA abundance. FIG. 6A shows schematics for a comparison of JFH-1-Nluc-WT and JFH-1-Nluc-GND, which has a GDD-to-GND mutation in NS5B, generating a non-functional viral polymerase. 2A/Ubi promotes “stop-carry on” translational recoding. FIG. 6B shows kinetics of nanoluciferase expression after transfection of in vitro-transcribed JFH-1-Nluc-WT and JFH-1-Nluc-GND RNAs into Huh7 cells, demonstrating that both RNAs can be translated and the wild-type genome can replicate. FIG. 6C shows measurements of off-target effects of vcircRNA junction-targeting siRNAs on the linear full-length viral RNAs. Cells were transfected with individual siRNA targeting the junction of different IRES-containing vcircRNAs and vcirc873/1151. At 24 hours post siRNA transfection, in vitro transcribed JFH1-NLuc-GND was introduced into cells, and NanoLuc assay was performed 24 hours later. FIG. 6D shows the effects of IRES-containing vcircRNAs on viral RNA abundance. Huh7 cells were transfected with control siRNA (siRNA-Ctrl), or pooled siRNAs targeting the junction sites of three IRES-containing vcircRNA (10/405, 14/362, 30/372) at a final concentration of 100 nM. At 1 day post transfection, cells were transfected with full-length viral RNA genome of HCV-10/405G-Hibit or HCV-25/1277-Hibit. After 1 day or 2 day post viral RNA genome delivery, viral RNA abundances in both 5′ UTR and Core regions were measured by qPCR. * p<0.05.

FIG. 7 shows quantification and effects of depletion of vcirc˜870/˜1151 on HCV RNA abundance. FIG. 7A shows absolute copy numbers of vcircRNAs quantified by droplet digital PCR. Total RNAs were extracted from mock- or HCV-infected (0.05 moi) Huh7 cells at 3 dpi. Divergent primers were used for detecting the vcircRNA (open circles), while convergent primers for linear viral RNA spanning the core region (triangles). “IVT RNA only” is in vitro-transcribed RNAs of JFH-1 RNA. FIG. 7B-C show depletion of vcirc873/1151. Huh7 cells were transfected with control siRNA (siRNA-Ctrl), or siRNA-a or -b targeting the junction site of vcirc873/1151 at a final concentration of 25 nM. At 24 hours-post-transfection, cells were infected with HCV. Infected cells were collected at 3 dpi, and abundances of vcirc˜870/˜1151 (FIG. 7B) and linear HCV RNA (FIG. 7C) were determined by qPCR. At least 4 biological replicates were used in qPCR. FIG. 7D shows the effects of siRNAs directed against vcircRNA 873/1151 on translation of HCV RNA carrying a replication defective polymerase gene and a luciferase reporter (JFH1-Nluc-GND).

FIG. 8 shows depletion of overexpressed vcircRNA 873/1151 by siRNAs. FIG. 8A is a schematic of the overexpression plasmid that contains two laccase inverted repeats to generate vcircRNA 873/1151. Huh7 cells were transfected with the plasmid. At 48 hours post transfection, RNA was isolated. RT-PCR was performed to verify the exogenous expression of vcircRNA using divergent primers flanking the junction. RT-PCR products were displayed on an agarose gel. FIG. 8B shows vcirc873/1151 overexpression in Huh7 cells first transfected with two siRNA targeting the junction sequence of vcirc873/1151 individually and then transfected with plasmid placcase-c873/1151. Circ873/1151 abundance was quantified by real-time PCR using divergent primers spanning the junction.

### DETAILED DESCRIPTION

Although DNA viruses were known to generate virus-derived circular RNAs by a back-splicing mechanism during polymerase II-mediated transcription, the generation of virus-derived circular RNAs from RNA viral genomes was not considered feasible as it was thought to destroy the viral RNA genome. However, the present disclosure shows for the first time that viral RNAs in cytoplasmic RNA viruses get circularized, not at the beginning but toward the exponential phase of viral RNA amplification, allowing the accumulation of sufficient full-length viral RNA to be packaged. Biochemical, genetic, and cell biological approaches were used to identify the mechanisms by which virus-derived circular RNAs are generated and their roles in infected and uninfected bystander cells. Rolling circle amplification was used to verify the presence of virus-derived circular RNAs. Additionally, depletion and over-expression studies showed pro-viral roles for virus-derived circular RNAs and revealed translation by IRES-containing virus-derived circular RNAs into novel proteins. Thus, provided herein are novel compositions and methods for depleting proviral virus-derived circular RNAs or for promoting anti-viral circular RNAs.

### Virus-Derived Circular RNAs and Polypeptide Products Thereof

As described herein, the virus-derived circular ribonucleic acids (RNAs) can be derived from RNA viruses (cytoplasmic or nuclear). As used throughout, the term RNA can encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

The term virus-derived circular RNA or vcircRNA is meant to encompass those circular RNAs or linear sequences thereof isolated from RNA viruses or those modeled after RNA isolated from or isolatable from RNA viruses. Virus-derived circular RNA is circularized when the 3′ terminus of an RNA sequence is covalently linked to the 5′ terminus of a contiguous upstream RNA sequence. Circular RNAs are more stable and less susceptible to degradation by exoribonucleases than linear RNAs.

Cytoplasmic RNA viruses from which circular RNA can be derived include, by way of example, flaviviruses (e.g., hepatitis C virus (types 1-7), Zika virus, West Nile virus, Dengue virus, yellow fever virus, and St. Louis encephalitis virus), coronaviruses (e.g., Severe Acute Respiratory Syndrome (SARS) virus, SARS-associated coronavirus (SARS-COV-2), MERS-CoV, HCoV-NL63, HCOV-229E, HCoV-OC43, and HCoV-HKU1), reoviruses (e.g., rotavirus or Colorado tick fever virus), enterovirus, picornaviruses (e.g., Coxsackievirus or rhinovirus) poliovirus, and hepatitis A), and togaviruses (e.g., Chikungunya virus).

Nuclear RNA viruses from which circular RNA can be derived include, by way of example, orthomyoviruses (e.g., influenza viruses) or retroviruses (e.g., HIV).

The virus-derived circular RNA as described herein can be modeled after any one of the genomes of the RNA viruses and synthesized by one of skill in the art using methods for polynucleotide synthesis. As shown herein, circular RNAs can be predicted from the viral genome by RNA-sequencing analysis (i.e., reads having two discontinuous mappings and containing a unique reading from 3′ to 5′ region spanning the junction). Virus-derived circular RNAs can be further identified by rolling circle amplification and revealed as a full copy of the circular RNA sequence and two flanking junction sites.

At least a subset of virus-derived circular RNA from a given virus genome include an internal ribosome entry site (IRES) and can be translated into a polypeptide product. In certain cases, the virus-derived circular RNA or its polypeptide product have proviral or anti-viral properties. One of skill in the art, according to the methods taught herein, can test the virus-derived RNA or its polypeptide product for such properties. At least a subset of virus-derived circular RNAs or polypeptide products have effects on infected cells, bystanders cells, or both infected cells and bystander cells. Thus, one of skill in the art, according to the methods taught herein, can test the virus-derived RNA or its polypeptide product for its effect on a given target cell type.

Agents that Block Proviral Functions or Promote Anti-Viral Functions

Once virus-derived circular RNAs and/or their polypeptide products are identified and their function determined, an agent can be used to block proviral functions of one or more virus-derived circular RNAs from RNA viruses. Such blocking agents can be small interfering RNAs (siRNAs) or a short hairpin RNAs (shRNAs). Alternatively, RNA-targeting endonuclease systems (e.g., CRISPR/Cas systems) can be used to target one or more virus-derived circular RNAs having a proviral function so as to disrupt the formation of the virus-derived circular RNA (for example, by editing or deleting the junction region so as to prevent circularization), to block the translation of the virus-derived RNA (for example, by editing or deleting the IRES so as to prevent translation), or to disrupt a proviral function of the virus-derived circular RNA (e.g., by editing or deleting an active region of the RNA or a polypeptide translated therefrom).

The CRISPR/Cas system, an RNA-guided nuclease system that employs a Cas endonuclease, can be used to edit the genome of RNA virus. The CRISPR/Cas system refers to a widespread class of bacterial systems for defense against foreign nucleic acid. CRISPR/Cas systems are found in a wide range of eubacterial and archaeal organisms. CRISPR/Cas systems include type I, II, and III sub-types. Wild-type type II CRISPR/Cas systems utilize an RNA-mediated nuclease, for example, Cas9, in complex with guide and activating RNA to recognize and cleave foreign nucleic acid. Guide RNAs having the activity of both a guide RNA and an activating RNA are also known in the art. In some cases, such dual activity guide RNAs are referred to as a single guide RNA (sgRNA).

As used herein, the term Cas9 refers to an RNA-mediated nuclease (e.g., of bacterial or archeal origin or derived from a bacterial or archeal nuclease). Exemplary RNA-mediated nucleases include the foregoing Cas9 proteins and homologs thereof. Other RNA-mediated nucleases include Cpf1 (See, e.g., Zetsche et al., Cell, Volume 163, Issue 3, p 759-771, 22 Oct. 2015), Cas13-based RNA editors, Cas-CLOVER (Li et al., Cas-CLOVER™: A High-Fidelity Genome Editing System for Safe and Efficient Modification of Cells for Immunotherapy. 2018 Precision CRISPR Congress Poster Presentation, Boston, MA) and homologs thereof.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chloroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi et al., RNA Biol. 2013 May 1; 10 (5): 726-737; Makarova et al., Nat. Rev. Microbiol. 2011 June; 9 (6): 467-477; Hou et al., Proc Natl Acad Sci USA 2013 Sep. 24; 110 (39): 15644-9; Sampson et al., Nature, 2013 May 9; 497 (7448): 254-7; and Jinek et al., Science 2012 Aug. 17; 337 (6096): 816-21. Variants of any of the Cas9 nucleases provided herein can be optimized for efficient activity or enhanced stability in the host cell. Thus, engineered Cas9 nucleases are also contemplated. See, for example, Slaymaker et al., Rationally engineered Cas9 nucleases with improved specificity, Science 351 (6268): 84-88 (2016)).

Small molecules can also be used to block a pro-viral function. Such molecules that target a specific RNA structure in a virus-derived RNA having a proviral function can be attached to an RNA degrading enzyme or portion thereof that retains the RNA degradation function. RNA degradation enzymes include endonucleases that cut RNA internally, 5′ exonucleases that hydrolyze RNA from the 5′ end, and 3′ exonucleases that degrade RNA from the 3′ end.

Similarly, if a virus-derived circular RNA itself or a polypeptide product translated therefrom has anti-viral functions, such RNA or polypeptide is an agent that promotes anti-viral functions. Additionally, a virus-derived circular RNA may be used to induce an immune response in a subject.

### Vectors, Nanoparticles, and Compositions

Virus-derived circular RNAs or nucleic acids encoding polypeptides translated therefrom encoding or agents that block proviral functions of the virus-derived circular RNA as described herein can be packaged in vectors. Such a vector can be chosen from viral vectors and non-viral vectors, plasmids, cosmids, and artificial chromosomes. By way of example, the vector can be a viral vector, such as a lentiviral vector or a retroviral vector. The vector optionally comprises nucleic acid sequences that encode transposases and/or nucleases. Non-viral vector examples include physical vectors such as electroporation and chemical vectors, such as a lipid nanoparticles.

Provided herein is a nanoparticle, such as a lipid nanoparticle, comprising a polymer layer and a virus-derived circular RNA, polypeptides translated from the RNA, an agent such as an siRNA or shRNA that blocks a proviral function of the virus-derived circular RNA, a vector (e.g., a vector comprising an RNA or related DNA, an agent that blocks a proviral function of the RNA or a nucleic acid that encodes the agent, or a gene editing system such as a CRISPR-Cas system), or a small molecule as described herein. As used herein, the term nanoparticle refers to a polymeric particle in the nanometer range. Optionally, the nanoparticle is a lipid nanoparticle with nucleic acids or small molecules adhered to or encapsulated therein. The lipids may comprise one or more lipids or amphiphilic compounds. For example, the particles can be liposomes, lipid micelles, solid lipid particles, or lipid-stabilized polymeric particles. The lipids can be made from one or a mixture of different lipids. Lipids are formed from one or more lipids, which can be neutral, anionic, cationic, or ionizable. For example, ionizable lipids can be positively charged during production, neutral in storage and in the blood, and revert to positive charge in lysosomes. In some embodiments, ionizable lipids may be composed of an amine moiety and a lipid moiety, and a cationic amine moiety and a polyanion nucleic acid interact electrostatically to form a positively charged liposome or lipid membrane structure. The liposomes can be unilamellar and/or multilamellar liposomes. By way of example, cationic liposomes can be formed from a composition of cationic lipids and phospholipids to form aggregates with macromolecules such as DNA and RNA.

Also provided are compositions comprising the virus-derived circular RNA, an agent that blocks a proviral function thereof, vectors, small molecules, or nanoparticles as described herein. Such compositions can be formulated as pharmaceutical compositions. The pharmaceutical composition can further comprise a carrier. The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water. Depending on the intended mode of administration, a pharmaceutical composition as described herein, can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the virus-derived circular RNA, agent that blocks a proviral function thereof, vector, small molecule, or nanoparticle described herein in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

### Methods of Use

The present disclosure provides a method of reducing a proviral effect of a virus-derived circular RNA of an RNA virus in a target cell by contacting a target cell with or administering to the target cell an agent that blocks the proviral function of the virus-derived circular RNA. The method optionally includes the step of identifying a virus-derived circular RNA having a proviral effect. Such virus-derived circular RNAs can be identified for example in silico as described in the Examples. The identified virus-derived circular RNAs can then be tested as described herein for proviral or antiviral functions.

The virus-derived circular RNA having a proviral or antiviral effect can be identified empirically according to the methods taught herein. For example, a target cell can be infected with an RNA virus or, in the case of a bystander cell, the target cell can be adjacent to or located near an infected cell or cells. The target cell can be contacted with the virus-derived circular RNA identified from the same RNA virus or the virus-derived circular RNA to be tested can be administered to the target cell. The target cell or cells can then be tested for proviral or antiviral effects, including for example, viral read counts in the infected cell. By way of example, an increase in viral read counts as compared to a control indicates the virus-derived circular RNA has a proviral function, whereas a decrease in viral read counts as compared to a control indicates the virus-derived circular RNA has an antiviral function.

If the virus-derived circular RNA has a proviral effect, the target cell can be contacted with or administered an agent that blocks the proviral function of the virus-derived circular RNA. Such agents are described herein and can include siRNA, an shRNA, an RNA-targeting CRISPR-Cas system, or a small molecule (e.g., a small molecule that targets the RNA and is optionally conjugated to an RNA degradation enzyme). By way of example, siRNAs against the junction site of a virus-derived circular RNA to knock down circRNA873/1151 (SEQ ID NO: 38) in hepatitis C include SEQ ID NO:19 and SEQ ID NO:20. Optionally the agent that blocks a proviral function of the virus-derived circular RNA is an siRNA that comprises a nucleotide sequence of a transcription regulatory sequence of the virus-derived circular RNA. Such an siRNA blocks the function of the transcription regulatory sequence.

Provided herein is a method of modulating an effect of a virus-derived circular RNA on one or more uninfected bystander cells by contacting the one or more uninfected bystander cells with a therapeutically effective amount of an agent that blocks a proviral effect of the virus-derived circular RNA on the one or more uninfected bystander cells or an agent that promotes an antiviral effect of the virus-derived circular RNA in the one or more uninfected bystander cells.

Also provided herein is a method of promoting an innate immune response by providing an immunogenic composition in a subject by administering to the subject a virus-derived circular RNA derived from a genome of an RNA virus or the polypeptide that is encoded by the virus-derived circular RNA. Similarly, provided herein is a method of promoting an innate immune response in a subject by administering to the subject an agent that blocks a function of a virus-derived circular RNA derived from a genome of an RNA virus and wherein the virus-derived circular RNA reduces in the subject an innate immune response to the RNA virus. The agent that blocks a function of the virus-derived circular RNA (i.e., inhibiting the innate immune response in the subject to the RNA virus) is optionally an siRNA, an shRNA, a RNA-targeting CRISPR-Cas system, or a small molecule (e.g., a small molecule that targets the virus-derived circular RNA and is conjugated to an RNA degradation enzyme or portion thereof).

Such methods of promoting an innate immune response can result in the subject being immune to future infections by the RNA virus or a subject that, if infected, has a lower grade infection that in the absence of administration of the RNA or agent that blocks the RNA function. The methods as described herein can be administered in conjugation with other vaccines and/or in conjunction with one or more adjuvants.

Also provided is a method of treating an RNA viral infection in a subject by detecting in a biological sample from the subject a virus-derived circular RNA or a polypeptide translated from a virus-derived circular RNA, wherein the virus-derived circular RNA is derived from a genome of an RNA virus; and administering to the subject a therapeutically effective amount of an antiviral agent. The antiviral agent is optionally an agent that blocks a function of the virus-derived circular RNA. For example, the antiviral agent can be an siRNA, an shRNA, a RNA-targeting CRISPR-Cas system, or a small molecule.

In the methods described herein the virus-derived circular RNA or polypeptide, antiviral agent or the like can be administered by a nanoparticle, a vector, or a composition.

In the treatment methods described herein, the virus-derived circular RNA, agents that block proviral effects of virus-derived RNAs, vectors comprising the virus-derived circular RNA or agents that block proviral effects of virus-derived RNAs, or small molecules (e.g., a small molecules that targets a virus-derived circular RNA, which is optionally attached to an RNA degradation enzyme), or nanoparticles or compositions comprising one or more of the RNAs, agents, vectors, or small molecules are administered in a therapeutically effective amount. As used herein, the term therapeutically effective amount or effective amount refers to an amount of an RNA, agent, small molecule, vector, nanoparticle, or composition as described herein, that, when administered to a subject, is effective, alone or in combination with additional agents, to treat a disease or disorder either by one dose or over the course of multiple doses. A suitable dose can depend on a variety of factors including the particular therapeutic, whether the therapeutic is used concomitantly with other therapeutic agents. Other factors affecting the dose administered to the subject include, e.g., the type or severity of the disease. For example, a subject having a severe hepatitis C infection may require administration of a different dosage as compared to a subject with a coronavirus infection or with a less severe hepatitis infection. Other factors that influence dosage and course of treatment include medical disorders concurrently or previously affecting the subject, the general health of the subject, the genetic disposition of the subject, diet, time of administration, and any other additional therapeutics that are administered to the subject. It should also be understood that a specific dosage and treatment regimen for any particular subject also depends upon the judgment of the treating medical practitioner. A therapeutically effective amount is generally also one in which any toxic or detrimental effects of the composition are outweighed by the therapeutically beneficial effects.

As used herein, administering or administration refers to the act of introducing, injecting or otherwise physically delivering a substance into a subject or a cell. Delivery to a subject can be by mucosal, intradermal, intravenous, intratumoral, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

Local or systemic treatment may be desired given the scope of infection. Administration to a subject can be via any of several routes of administration, including orally, parenterally, intramucosally, intravenously, intraperitoneally, intraventricularly, intramuscularly, subcutaneously, intracavity or transdermally. Administration can be achieved by, e.g., topical administration, local infusion, injection, or by means of an implant.

As used throughout, a subject can be a vertebrate, more specifically a mammal (e.g., a human, horse, cat, dog, cow, pig, sheep, goat, mouse, rabbit, rat, and guinea pig). The term does not denote a particular age or sex. Thus, adult, newborn and pediatric subjects, whether male or female, are intended to be covered. As used herein, patient or subject may be used interchangeably and can refer to a subject with or at risk of developing a disease or disorder. A subject can be at risk of developing an RNA viral infection, for example, upon an exposure to another subject with an infection, a subject who is a carrier of the virus, or other source. The term patient or subject includes human and veterinary subjects. In any of the methods provided herein, the subject can be a subject diagnosed with infection by an RNA virus.

As used herein, the terms treatment, treat, or treating refers to a method of reducing one or more of the effects of the disease or disorder or one or more symptoms of the disease or disorder, for example, an RNA viral infection in the subject. Thus, in the disclosed methods, treatment can refer to a 10%, 20%, 30%, 40%, 50%, 60%, 70%, 80%, 90%, or 100% reduction in the severity of the infection. For example, a method for treating hepatitis C is considered to be a treatment if there is a 10% reduction in one or more symptoms of the infection in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disorder or symptoms of the disease or disorder.

Publications cited herein and the material for which they are cited are hereby specifically incorporated by reference in their entireties.

### EXAMPLES

The disclosure may be better understood by reference to the following non-limiting examples.

### Example 1: Cell Culture, HCV Infection, and RNA Extraction

The human hepatocarcinoma cell line Huh7 was maintained in Dulbecco's modified Eagle's medium (DMEM) (Invitrogen, Waltham, MA) supplemented with 10% heat inactivated fetal bovine serum (FBS), pen/strep and non-essential amino acids. Cells were grown in an incubator with 5% CO2 at 37° C. For infection, six well-plates were seeded with 2.5×105 Huh7 cells per well. On the next day, supernatants were removed, and cells were infected with HCV JFH-1 viral stock at MOI of 0.1 for 3 hours. The inoculum was removed and replaced with 2 mL of DMEM. At 48 or 72 hours post-infection, total RNA from infected and non-infected cells was extracted using TRIzol reagent (Invitrogen, Waltham, MA), following the manufacturer's instructions. Genomic DNA was removed in-column by Turbo DNase I treatment (ThermoFisher, Waltham, MA) using RNA concentrate and clean kit (ZYMO Inc., Irvine, CA).

### Example 2: Sample Preparation for RNA-Seq

RNA samples with an RNA integrity ≥9 were subjected to construct RNA-Seq libraries by using True-seq Stranded Total RNA Library Kit (Illumina Inc. USA, San Diego, CA). These libraries were then sequenced on the Illumina sequencing platform (NovaSeq 6000 platform) and 150 bp/125 bp paired-end reads were generated. Illumina sequencing and Quantity Control (QC) of the raw data were performed at Stanford Genomics (Stanford, CA, USA).

### Example 3: In Silico Identification of Viral Circular RNAs (vcircRNA)

The Galaxy tool, a web-based platform for data intensive biomedical research, was used to perform Q30 and adaptor trimming on the raw data, and subsequently to align the reads with JFH-1 reference genome (GenBank No. AB047639). An expectation value cutoff of 10-5 was used in the blastn. On the blast-hit table, focus was given to the reads that were mapped to two discontinuous regions on the viral genome. These candidate reads were then subjected to an in-house pipeline searching for junction reads that have the correct alignment of the four anchor points on the viral genome according to the back-splicing mechanism. Finally, junction reads demonstrating a 5′ most and a 3′ most end as splice sites were considered as predicted vcircRNA reads.

### Example 4: Validation of HCV circRNAs Using Rolling Circle Amplification and PCR

One μg RNA was reverse transcribed using a Superscript III kit (Invitrogen, Waltham, MA) accordingly to the manufacturer's instruction. A reverse primer specific to the 5′ junction site of the circRNA was added in the reaction to increase the yield of the cDNA of circRNAs. Subsequently, nested PCR was performed using 0.5 μL cDNA and two sets of divergent primers flanking the junction site of the specific circRNA. For amplifying IRES-containing circRNAs, the following primer pairs were used: (1) forward4: TGAGCACAAATCCTAAACCTCA (SEQ ID NO:1) and reverse4: TTCCTCACAGGGGAGTGATT (SEQ ID NO:2) and (2) forward5: CAAATCCTAAACCTCAAAGAAAAACC (SEQ ID NO:3) and reverse5: CTCACAGGGGAGTGATTCATGG (SEQ ID NO:4). For the most abundant circRNAs with 5′ in proximity to 860 nt and 3′ to 1150 nt (circRNA˜860/˜1150), the following primer pairs were used: (1) forward9: CCAGTCTCGCCAAACATGGCTGT (SEQ ID NO:5) and reverse9: CACCATGTAGCTGCTACTGGTATTCTTCA (SEQ ID NO:6) and (2) forward10: GACGCACATCGATATGGTTGTGATGT (SEQ ID NO:7) and reverse10: GGAACGGTGATGCAGGACAACA (SEQ ID NO:8).

The cycling condition was set as follows: 98° C. 1 min, 30 cycles of 98° C. 10 sec, 56° C. 15 sec, 72° C. 30 sec and 72° C. 5 min. PCR products were then separated in 1% agarose gel. The resulting DNA band was extracted by Qiagen gel purification kit and then cloned into pCR4 Blunt-TOPO vector (Invitrogen, Waltham, MA). Colonies were sent for sequencing and blasted to the HCV strain JFH-1 sequence. A few circRNAs were then validated from those colonies, which contained either two copies of circRNA sequence or one copy of circRNA sequence with two repeated junction reads at both ends.

### Example 5: HCV circRNA and Viral mRNA Quantification by qPCR

One μg RNA was used to prepare cDNA by using a High-Capacity RNA-to-cDNA Kit (Applied Biosystems, Waltham, MA). qPCR was performed using the PowerUP SYBR green PCR mix in a 96-well format in the CFX96 qPCR machine according to the manufacturer's instructions (BioRad, Hercules, CA). The following primer pair were used for HCV viral mRNA: forward CCCTATATGGGAATGAGGGACT (SEQ ID NO:9) and reverse CGTTAGGGTGTCGATGACTTTAC (SEQ ID NO:10). The following primer pair was used for the most abundant HCV circRNAs ˜860/˜1150: forward GACGCACATCGATATGGTTGTGATGT (SEQ ID NO:7) and reverse CACCATGTAGCTGCTACTGGTATTCTTCA (SEQ ID NO:6). The following primer pair was used for HPRT1: forward CGAGATGTGATGAAGGAGATGG (SEQ ID NO:11) and reverse TTGATGTAATCCAGCAGGTCAG (SEQ ID NO:12). Transcript abundance was calculated using the delta-cT method.

### Example 6: Droplet Digital PCR (ddPCR)

Droplet Digital PCR was performed using Bio-Rad's QX200 Droplet Digital PCR system (Stanford Genomics, Stanford, CA). Briefly, 40 ng of cDNA were mixed with 10 μL of QX200ddPCR EvaGreen supermix and 125 nM forward and reverse primers (described above). The reaction was then dispensed into sample wells in the DG8 cartridge so that droplets were generated according to the manufacturer's instruction. The droplets were then transferred to a 96 well plate and run on a thermal cycler using the following conditions: 95° C. for 5 min, 42 cycles of 98° C. for 30 sec and 59° C. for 1 min, and 4° C. for 5 min and 90° C. for 5 min. After PCR was completed, the plate was transferred in to a QX200 droplet reader and analyzed by QuantaSoft Analysis Pro software (Bio-Rad, Hercules, CA). Both HCV viral RNA and HCV circRNA were quantitated separately for each sample, with at least 3 biological repeats.

### Example 7: Construction of Placasse2-Split-GFP Plasmids Inserted with circRNA Sequence

To replace encephalomyocarditis virus-internal ribosomal entry site EMCV-IRES with the sequence (5′-3′) of HCV circRNA10/405, two fragments were amplified from placcase2-splitGFP (a gift from Dr. Jeremy E. Wilusz (University of Pennsylvania)) with the following primer pairs: forward 1 CTTGGTACCGAGCTCGGATCCACTAGTCCAGTGTGGTGGAAT (SEQ ID NO:13), reverse 1 TTATTAATTAAGTCGACTGCAGAATTCAGATCC (SEQ ID NO:14); forward 2 GTCCTGCAGGGTATGGTGAGCAAGGGCGAGGAGCT (SEQ ID NO:15); reverse 2 AACGGGCCCTCTAGACTCGAGCGGCCGCCTGAGGTGCCAC (SEQ ID NO:16). A third fragment was amplified from pCR4 Blunt-TOPO cloned in with HCV circRNA10/405. The following primers were used to include two restriction enzyme sites PacI and SbfI at its 5′ or 3′ end: forward GCAGTCGACTTAATTAATAATAGGGGCGACACTCCGCC (SEQ ID NO:17), reverse CTCACCATACCCTGCAGGACGTCTTCTGGGCGACGGTTG (SEQ ID NO:18). The vector placcase2-splitGFP was then digested with BamHI and XhoI.

Next, the above three amplified fragments and the digested vector were subjected to the InFusion Cloning (Takara Bio., Kusatsu, Shiga, Japan). The resulting plasmid placcase-splitGFP-10/405 was later digested with PacI and SbfI and then used in the InFusion Cloning to generate plasmids inserted with the individual sequences of HCV circRNA30/372, circRNA14/362, circRNA26/369, and circ873/1151. The following primer pairs were used: for circRNA30/372 (forward TCTGCAGTCGACTTACATGAATCACTCCCCTGTGA (SEQ ID NO:39) and reverse TTGCTCACCATACCCGTTTTTCTTTGAGGTTTAGGATTTG (SEQ ID NO:40)); for circRNA14/362 (forward TCTGCAGTCGACTTAATTAAAGGGGCGACACTCCGCCATG (SEQ ID NO:41) and reverse TTGCTCACCATACCCAGAGGTTTAGGATTTGTGCTCATGGT (SEQ ID NO: 42)); for circRNA26/369 (forward TCTGCAGTCGACTTACCGACATGAATCACTCCCCT (SEQ ID NO:43) and reverse TTGCTCACCATACCCTTTCTTTGAGGTTTAGGATTTGTG (SEQ ID NO:44)); and for circ873/1151 (forward TCTGCAGTCGACTTATTGCTGGCCCTGTTGTCC (SEQ ID NO: 45) and reverse TTGCTCACCATACCCGGTGGCGGACATCACAACC (SEQ ID NO: 46)).

For transfection, 4.5×105 Huh7 cells per well were seeded in 6-well plates. On the following day, 1.5 μg of each plasmid were transfected using lipofectamine 3000 according to manufacturer's protocol. Cells were visualized under confocal microscopy 2 days post-transfection.

### Example 8: Small Interfering RNA Experiments with JFH-1 Infection

The following siRNAs against the junction site of circRNA were used to knock down circRNA872/1150: sense siRNA-a CGCCACCUUGCUGGCCCUGUUdTdT (SEQ ID NO:19) and sense siRNA-c GCCACCUUGCUGGCCCUGUUGdTdT (SEQ ID NO:20). The siRNA duplexes were formed by combining sense and their corresponding antisense strands in 1×siRNA Buffer (Dharmacon, Lafayette, CO) as described previously. Chen et al. (2020), Host-derived circular RNAs display proviral activities in Hepatitis C virus-infected cells, PLOS Pathog. 16 (8): e1008346. As a negative control siRNA, the following oligonucleotides were used: sense GAUCAUACGUGCGAUCAGAdTdT (SEQ ID NO:21) and antisense UCUGAUCGCACGUAUGAUCdTdT (SEQ ID NO:22). For transfection, 3×105 Huh7 cells per well were seeded in 6 well plates. The following day, 50 nM of siRNA duplexes were transfected using Dharmafect I (Dharmacon, Lafayette, CO). After overnight incubation at 37° C., cells were infected with JFH-1 at a MOI of 0.1. Total RNA was extracted 3 days post-infection. depletion of circRNA872/1150 and JFH-1 viral mRNA level was assessed by real-time qPCR.

### Example 9: Generation of Mutant JFH-1 DNA Infectious Clone Containing a HiBiT Tag Inserted in the 5′ UTR

To insert a HiBiT tag and a mutation T32G, an upstream fragment of 125 bp was amplified from the wildtype pJFH-1 plasmid (infectious clone from Dr. Karla Kirkegaard lab (Stanford, CA)) by nested PCR with the following primers: first forward AAAAGCAGGCTACTCGAATTCTAATACGACT (SEQ ID NO:23), the first reverse primer AACAGCCGCCAGCCGCTCACGGGAGTGATTCCTGGCGGAGT (SEQ ID NO:24), and then the second reverse primer AGTTAGCTAATCTTCTTGAACAGCCGCCAGCCGCT (SEQ ID NO:25). A downstream fragment of 2951 bp was amplified with the forward primer TTCAAGAAGATTAGCTAACTGTGAGGAACTACTGTCTTC (SEQ ID NO:26) and the reverse primer CACGCGATGCCATCGCGGCC (SEQ ID NO:27). These two fragments were then included in the InFusion Cloning (Takara Bio, Kusatsu, Shiga, Japan), together with pJFH-1 plasmid digested with NotI and EcoRI. The resulting infectious clone was named pJFH1-HiBiT-Gga. To generate pJFH1-HiBiT-Tga, a similar Infusion cloning strategy was used with a different upstream fragment that was amplified by nested PCR, with forward primer AAAAGCAGGCTACTCGAATTCTAATACGACT (SEQ ID NO:23) and reverse primers CGCTCACGGGAGTGATTCATGGCGGAGTGTC (SEQ ID NO:28), GCCGCCAGCCGCTCACGGGAGTGATT (SEQ ID NO:29), and AGTTAGCTAATCTTCTTGAACAGCCGCCAGCCGCTC (SEQ ID NO:25).

### Example 10: In-Vitro Synthesized RNA and Transfection

Plasmids pJFH-1, pJFH-1-Gga-HiBiT, pJFH-1-Tga-HiBiT were linearized and transcribed using the T7 MEGAscript kit (Ambion, Austin, TX), according to the manufacturer's instructions. Their corresponding PCR products (226 nt-Gga-Hibit, 226 nt-Tga-Hibit, 444 nt-Gga-Hibit and 444 nt-Tga-Hibit) were amplified with primer pairs, forward ATCGAAATTAATACGACTCACTATAGGACCTGCCCCTAATAGGGGC (SEQ ID NO:30) and reverse226 GCCCTCTAGACTCGAACCCAGTCTTCCCGGCAA (SEQ ID NO:31) or reverse444 TTAACGTCTTCTGGGCGACGGTT (SEQ ID NO:32), and subsequently transcribed in vitro. Huh7 cells in 6 well plates were transfected with 2 μg of each in-vitro transcribed (IVT) RNA using the TransIT mRNA transfection kit (Mirus Bio, Madison, WI). After 18 hours or 24 hours of incubation, cells were harvested for HiBiT lytic nanoluciferase assay (Promega, Madison, WI) and HiBiT western blot assay (Promega, Madison, WI).

### Example 11: Examination of Functional Roles for HCV Derived vcircRNAs During the Viral Life Cycle

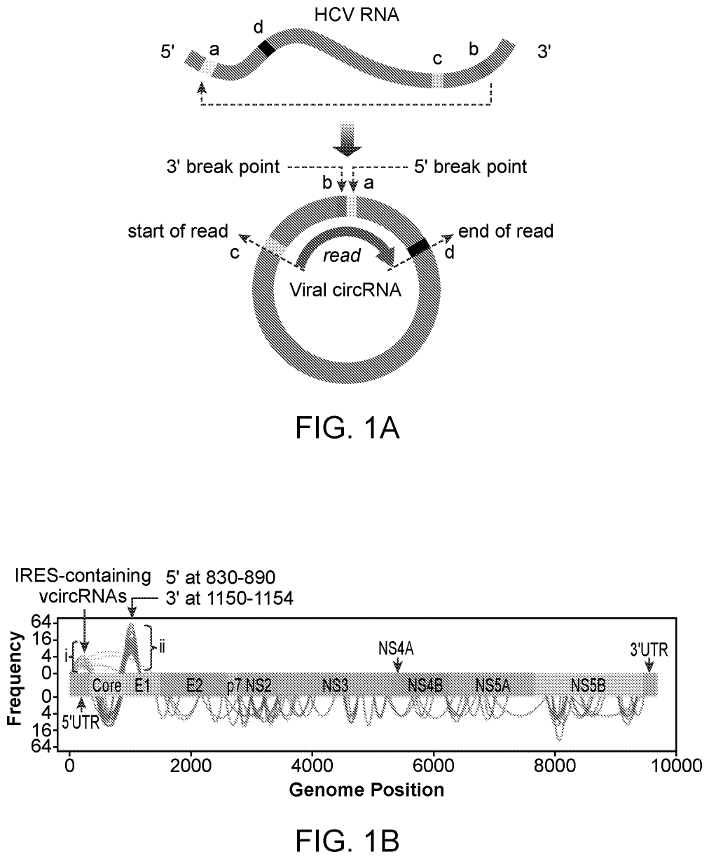

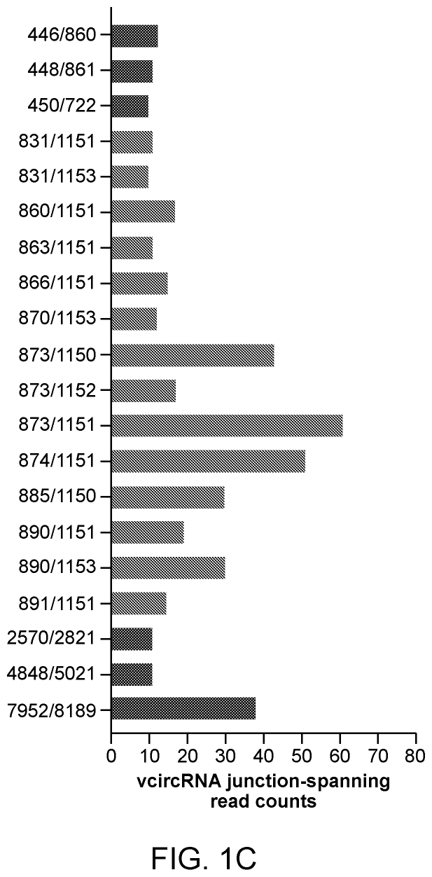

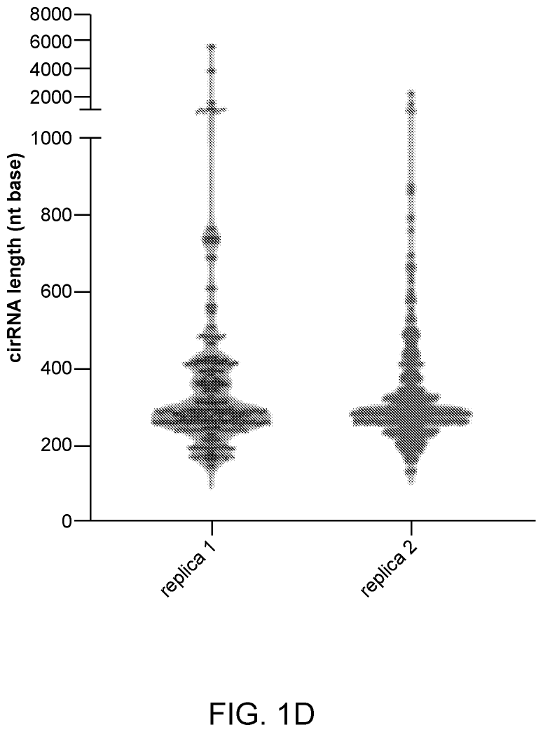

A study was conducted to identify any host circRNAs whose abundances were altered during hepatitis C virus (HCV) infection of liver cells. Upon interrogation of RNase R59 treated circRNA libraries used to identify these cellular circRNAs, hundreds of putative vcircRNA species derived from the 10,000-nucleotide HCV genome. During the process of alignment, de novo virus-derived junction-spanning reads were discovered, based on a defined order of four nucleotide positions that align discontinuously to the viral genome (FIG. 1A). Although the vast majority of total viral reads were mapped to continuous regions on the viral genome, consistent with their templating from linear viral sequences, approximately 1% of the reads contained such novel junction sequences. Most of these junction reads (97.7%) were derived from the positive strand of the RNA genome. Although the 5′ and 3′ breaks points in each junction were distributed across the entire HCV genome, their locations were not random (FIG. 1B). One cluster of reads was observed at the 5′ end of HCV, containing the viral internal ribosome entry site (IRES) and the N-terminal viral core sequences (shown as “i” in FIG. 1B). The most abundant cluster of predicted vcircRNAs (shown as “ii” in FIG. 1B) had 5′ breakpoints between positions 830 to 890 in the viral core coding sequence and 3′ breakpoints between nucleotide positions 1151 to 1154 in the envelope E1 coding sequence. The list of the top predicted vcircRNAs is shown in FIG. 1C. The sizes of predicted vcircRNAs range from 130 nts to larger 74 than 2000 nts, with an average size of 200-300 nts (FIG. 1D).

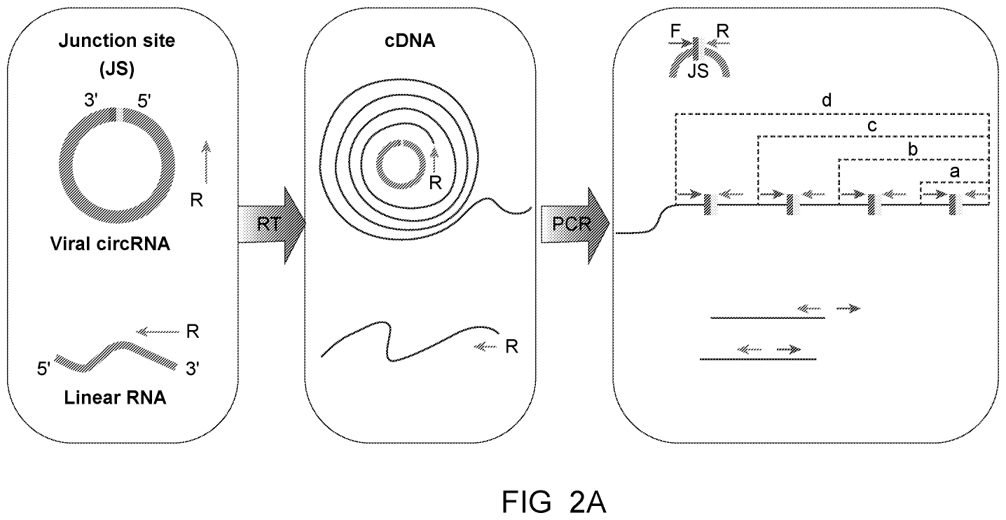

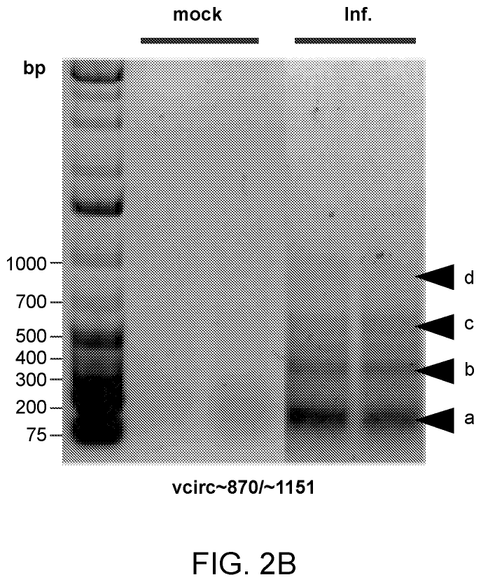

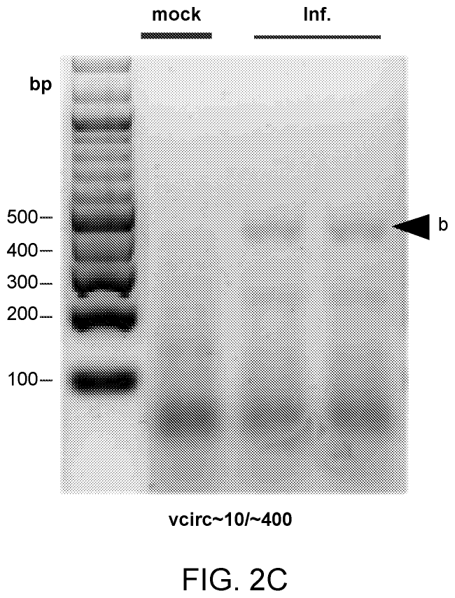

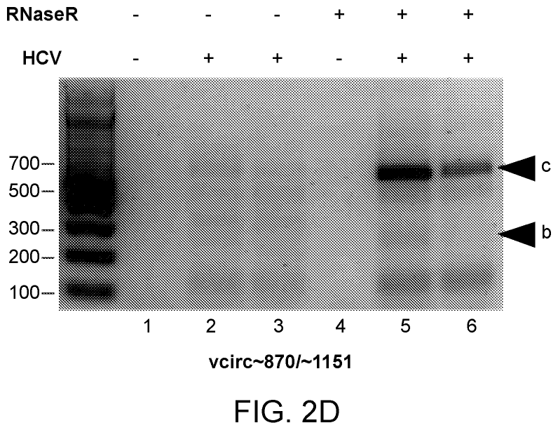

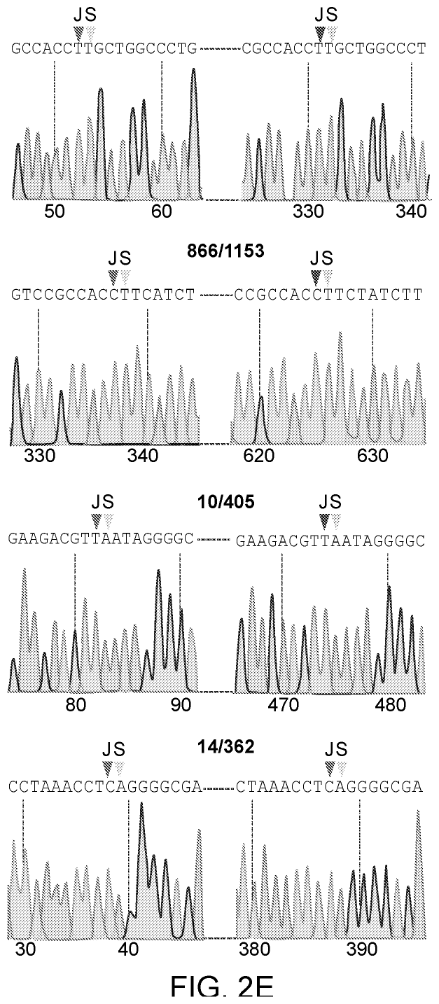

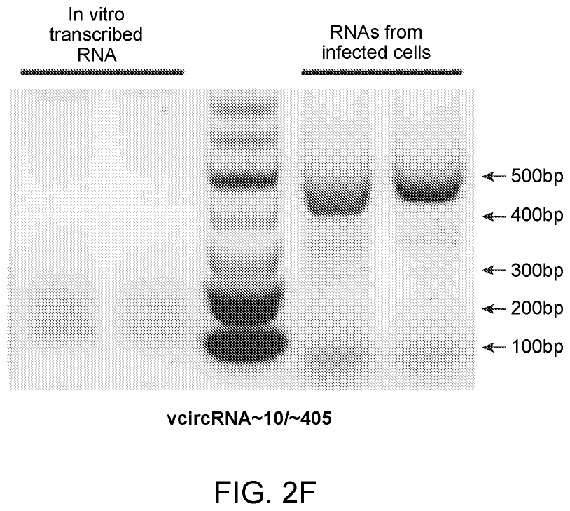

Although the presence of discontinuous junction sequences suggested their derivation from vcircRNAs, this needed to be experimentally verified. This was done by reverse transcriptase-mediated rolling circle amplification (Acevedo and Andino, Library preparation for highly accurate population sequencing of RNA viruses. Nat Protoc 9, 1760-1769 (2014). FIG. 2A describes the rolling-circle amplification strategy employed to verify several members of the most abundant ˜870/˜1151 putative vcircRNA cluster and members of the IRES-containing ˜10/˜400 putative vcircRNA cluster. Briefly, a reverse primer binding to the 5′ sequences of the predicted junction site in vcircRNAs was used to initiate the formation of rolling circle cDNA products. These products, predicted to contain multiple iterated sequences, were amplified by PCR using primers that flanked the novel junction but were divergent on the linear viral sequence (FIG. 2A). Multiple rounds of transcription of vcircRNAs will result in PCR products of various length that contain multiple junction sites, denoted as a, b, c, and d (FIG. 2A). In contrast, linear RNAs should not yield any products in the PCR reaction (FIG. 2A). Indeed, PCR fragments predictive of harboring multiple junction sequences were identified in infected cells, but not in mock-infected cells (FIG. 2B,C). No specific PCR products were amplified from in vitro-transcribed RNAs (FIG. 2F), strengthening the argument that the vcircRNAs were generated in infected cells and did not represent PCR template switching or other artifacts. In addition, the rolling circle amplification of the abundant vcircRNA ˜870/˜1151 was resistant to RNase R treatment, (FIG. 2D), further arguing that it represents a vcircRNA. Finally, sequencing of cloned PCR products revealed complete, iterated sequences of eight vcircRNAs in the abundant ˜870/˜1151 vcircRNA cluster and six in the IRES-containing ˜10/˜405 vcircRNA cluster (examples are shown in FIG. 2E). Each viral sequence contained at least two junction sites flanking one complete vcircRNA sequence from 5′ to 3′ break point (FIG. 2E). So far, 14 out of 14 predicted vcircRNAs have been verified by rolling circle amplification, suggesting that most of the predicted vcircRNAs are bona fide vcircRNAs.

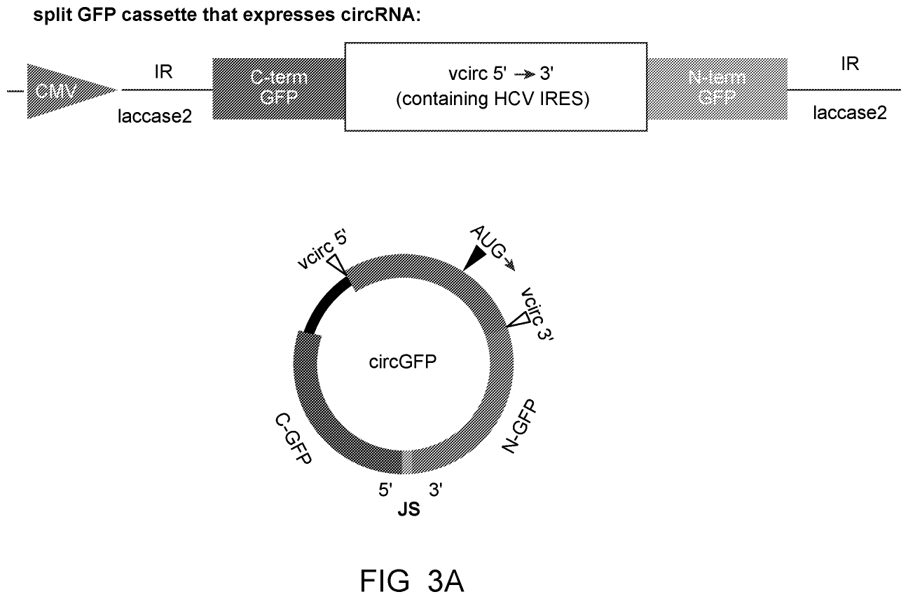

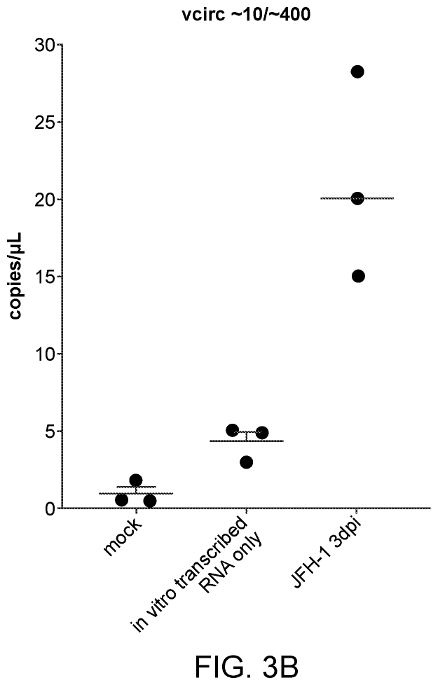

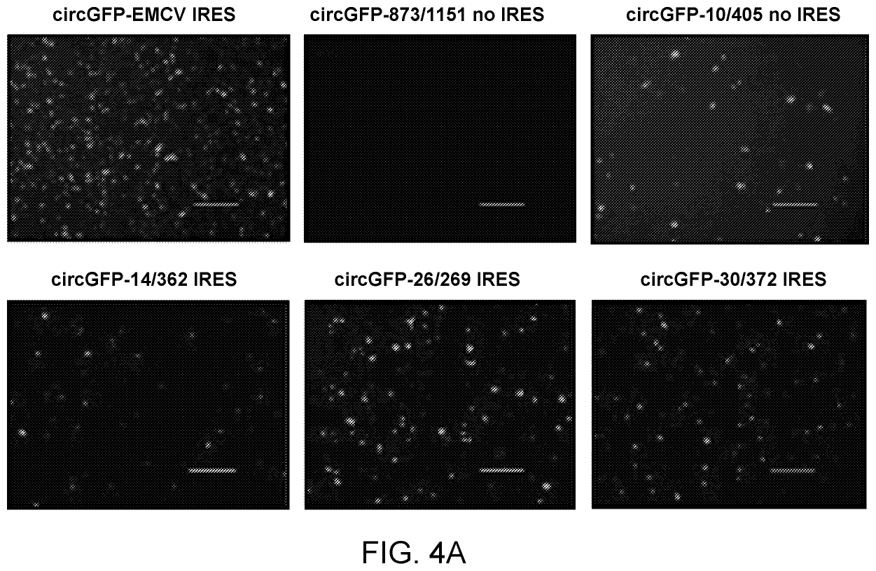

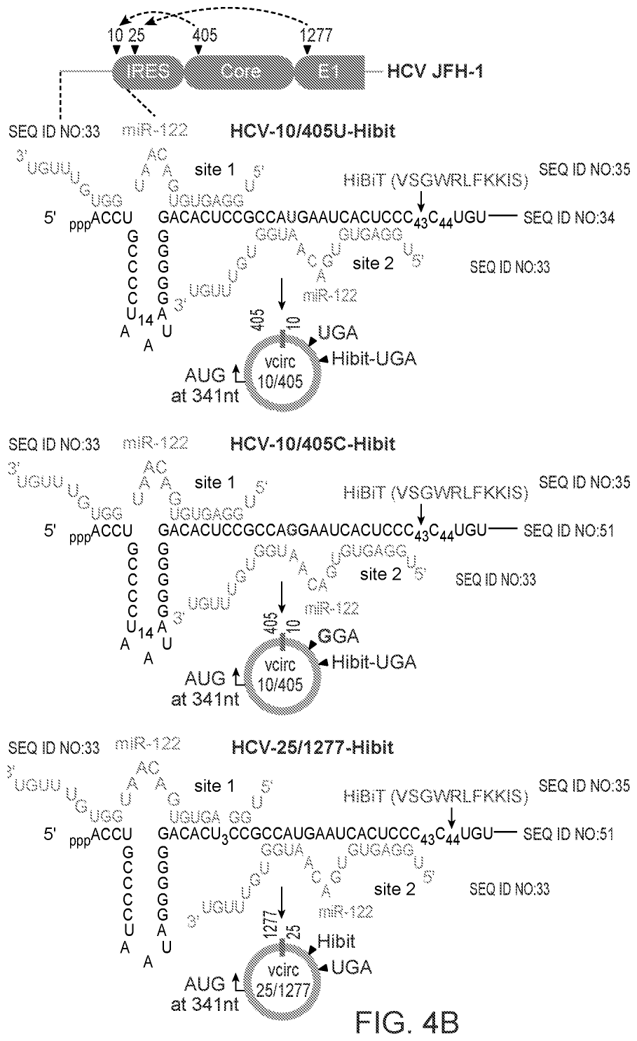

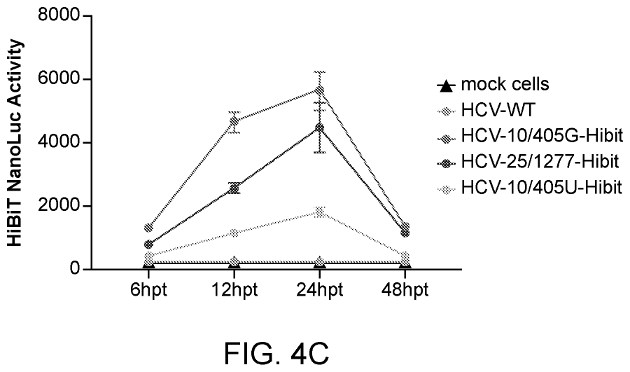

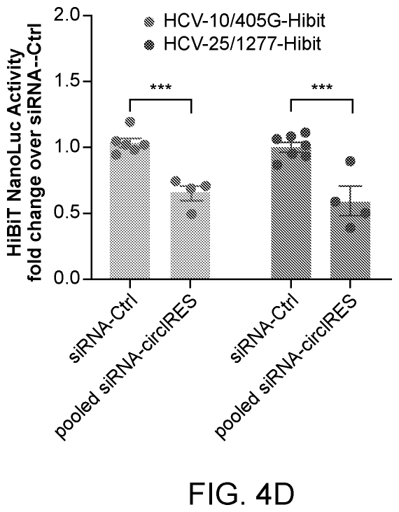

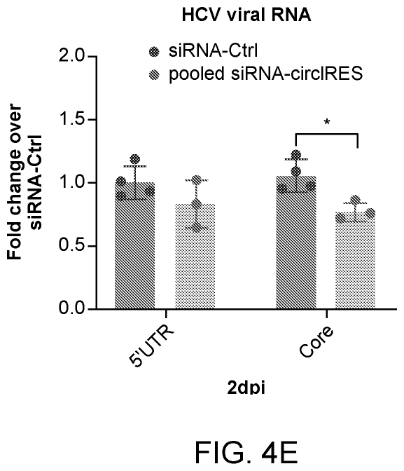

Synthetic circRNAs can be translated when they contain the IRES of encephalomyocraditis virus (EMCV). Thus, whether the HCV IRES-containing vcircRNAs represent a novel and natural class of vcircRNAs that can be translated into novel proteins was further investigated. First, the circularity of several members of the cluster, vcircRNA 10/405, vcircRNA 26/369, vcircRNA 30/372 and vcircRNA 14/362, was verified by rolling circle amplification. Secondly, whether these HCV IRES-containing circRNAs could mediate translation was investigated, when expressed from a split-GFP plasmid, in which the C-terminal coding sequence of GFP is placed before its N-terminal sequence, separated by the individual vcircRNA sequences that contain the HCV IRES (FIG. 3A). This cassette was flanked by two inverted Drosophila lacasse2 repeats, which efficiently undergo back-splicing events, to facilitate the circularization of the linear transcript, thus allowing IRES-mediated production of full-length GFP in transfected cells (Kramer et al., Combinatorial control of Drosophila circular RNA expression by intronic repeats, hnRNPs, and SR proteins. Genes Dev 29, 2168-2182 (2015)) (FIG. 4A). As expected, EMCV IRES-containing circRNAs produced GFP in a significant percentage of the transfected cells (FIG. 3A). In contrast, the 873/1151 HCV sequence, which does not contain an IRES like many other vcircRNAs, did not promote the synthesis of GFP (FIG. 4A). Satisfyingly, all IRES-containing HCV sequences expressed GFP (FIG. 4A), demonstrating that the HCV IRES is active when placed into a circRNA. Although some of the IRES-containing vcircRNAs are longer, all of them contain at least nucleotides 42-356 and are predicted to use the AUG at nucleotide 341 that is normally used for the translation of the HCV core protein. The proteins encoded by these vcircRNAs, depending on the location of their junction sequences, are predicted to contain the first 7-21 amino acids of core protein, followed by a novel sequence encoded from their 5′ break points until a stop codon is met.

The subsequent task was to determine whether the IRES-containing vcircRNAs were translated when produced from the HCV genome in infected cells. The IRES-containing vcircRNAs constitute only approximately 0.25% of the linear HCV RNA during viral infection, as determined by digital droplet PCR (FIG. 3B), and appropriate antibodies to the novel products are not yet available. Two different IRES-containing vcircRNAs, vcircRNA 10/405 and the larger vcircRNA 25/1277, were chosen to monitor the production of the predicted proteins. To enable detection of these predicted products, a sequence encoding a small peptide tag of 11 amino acids, called HiBiT (see Schwinn et al., CRISPR-Mediated Tagging of Endogenous Proteins with a Luminescent Peptide. ACS Chem Biol 13, 467-474 (2018)) was inserted downstream of the 5′ junction sequence in three different HCV genomes (FIG. 4B). As a part of the luminescent NanoBiT enzyme, HiBiT can be quantified as nanoluciferase activity (see Schwinn et al., CRISPR-Mediated Tagging of Endogenous Proteins with a Luminescent Peptide. ACS Chem Biol 13, 467-474 (2018); Oh-Hashi et al., Application of a novel HiBiT peptide tag for monitoring ATF4 protein expression in Neuro2a cells. Biochem Biophys Rep 12, 40-45 (2017)), making it a sensitive method for detecting very low-abundance translation events. Care was taken not to disrupt the essential microRNA binding sites in this region of the HCV RNA (see Jopling et al, Modulation of hepatitis C virus RNA abundance by a liver-specific MicroRNA. Science 309, 1577-1581 (2005)). The placement of the HiBiT tag allows HiBIT to be exclusively expressed from IRES-containing vcircRNAs.

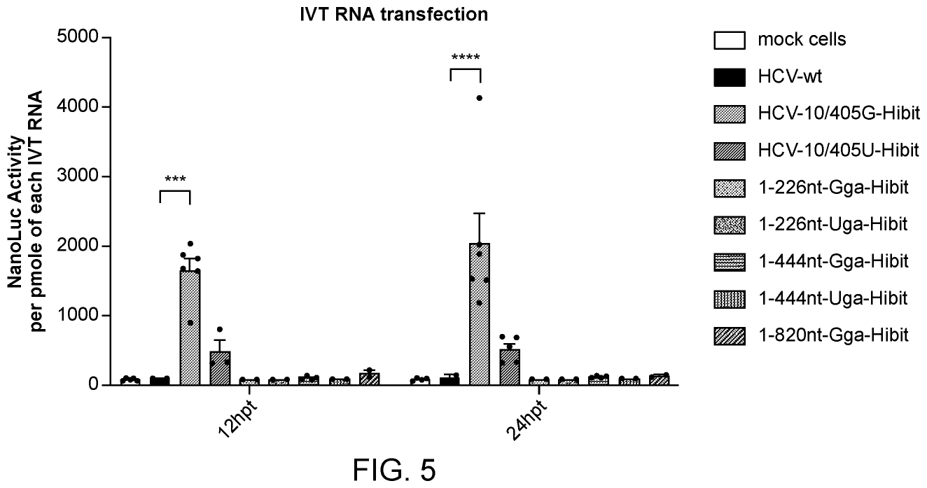

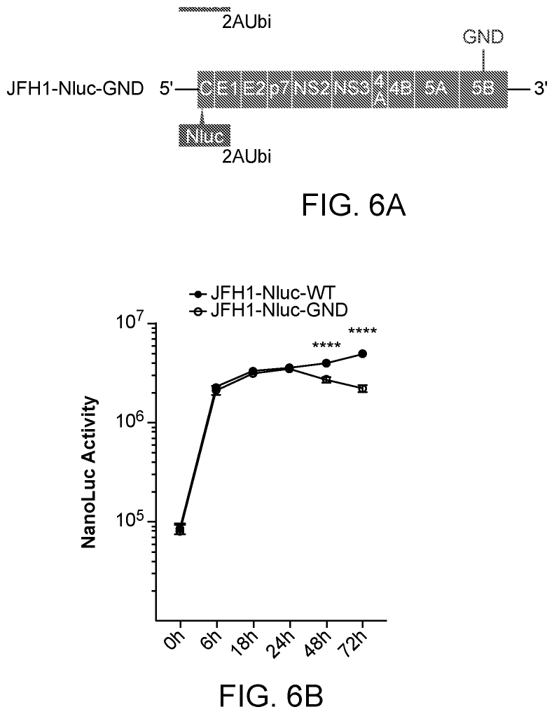

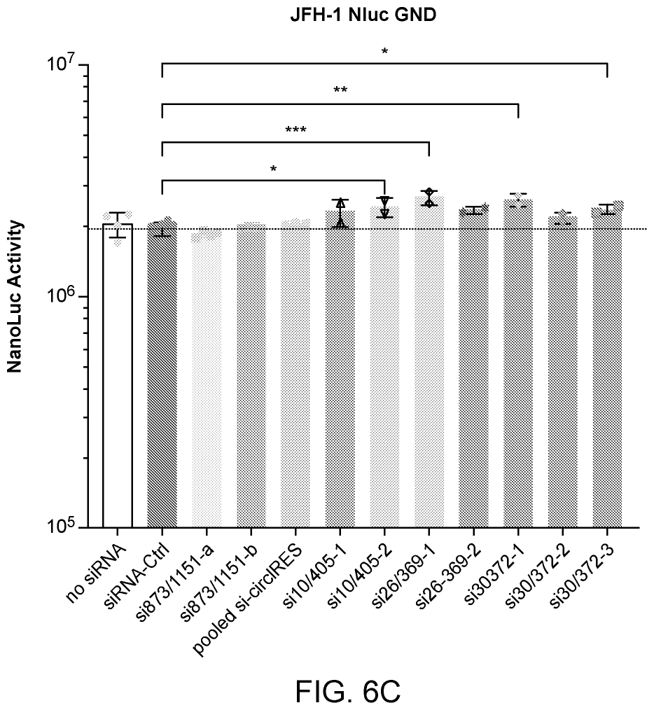

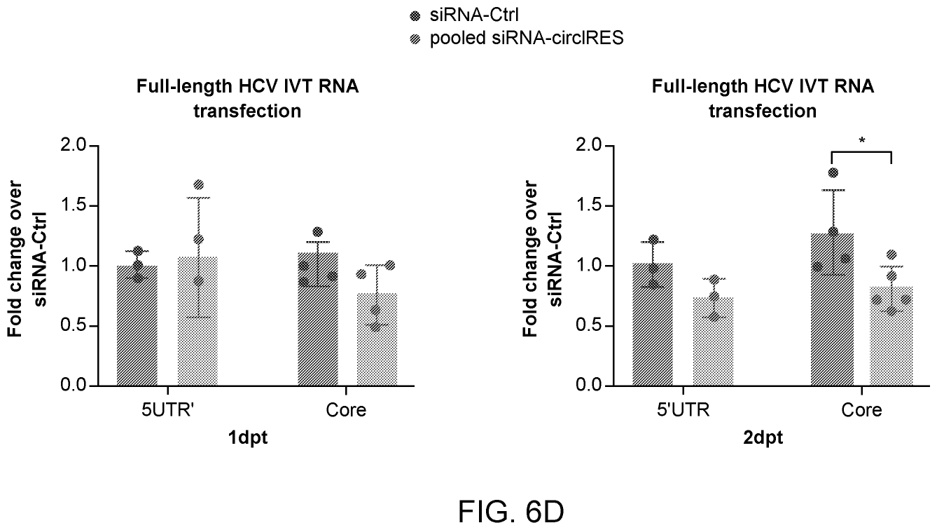

Three variant genomes were constructed to test the translatability of the HCV ˜10/˜405 vcircRNAs: HCV-10/405G-Hibit, predicted to generate a novel core-fusion-HiBiT peptide should HCV-10/405 circles be formed, and HCV-10/405U-Hibit, which creates a stop codon before HiBiT and should abrogate formation of a HiBiT-containing product. A third variant, HCV-25/1277-Hibit, was created to test the translatability of IRES-containing vcirc25/1277, which, interestingly, predicts a different reading frame through the nominally untranslated region (FIG. 4B). Full-length viral RNAs were transfected into Huh7 cells and HiBiT activities were measured post transfection. Unlike the HiBiT-lacking wildtype HCV, transfection of either the HCV-10/405G-Hibit or HCV-25/1277-Hibit genomes gave rise to significant luminescence (FIG. 4C). This result indicates that vcirc 10/405 and vcirc25/1277 can be translated when expressed from full-length viral RNA in infected cells. HCV-10/405U-Hibit mutant-transfected cells also exhibited luminescence, but to a much lesser extent than HCV-10/405G-Hibit. It is possible that HiBiT was expressed from other vcircRNA(s) in the IRES-circRNA cluster (FIG. 1B), or that viral linear RNA was translated in an IRES-independent manner. To test the latter possibility, linear RNA fragments from the 5′ end of HiBiT-inserted mutants were generated, and their translation examined after transfection into cells. These HiBiT-containing linear RNAs yielded only background luminescence (FIG. 5). To further test the hypothesis that the HCV-HCV-10/405G153 Hibit and 25/1277-Hibit vcircRNAs are translated, whether siRNAs directed to the junction sequences of IRES-vcircRNAs reduced luminescence was tested. Indeed, the IRES-vcircRNA targeting siRNAs diminished HiBiT accumulation (FIG. 4D). To test for off-target effects of the siRNAs on linear HCV RNAs, a replication-deficient JFH-1 viral genome with a nanoluciferase insertion (JFH1-NLuc-GND) was generated (FIG. 6A, B). FIG. 6C shows that the employed siRNAs did not diminish the translation of the replication-deficient HCV RNAs. Hence, the observed reduction in HiBIT expression from variant viruses by specific siRNAs against vcircRNAs supports the conclusion that IRES-containing vcircRNAs can be translated into novel products in HCV-infected cells.

Importantly, a decreased viral RNA abundance was observed after siRNA-mediated depletion of the vcircRNAs at 2 days after HiBiT-containing viral RNA transfection (FIG. 6D), suggesting IRES-containing vcircRNAs may have a role in promoting viral replication. To further test this hypothesis, IRES-vcircRNAs were depleted by siRNAs, followed by infection with wildtype JFH1 HCV. Core-containing viral RNA abundances were diminished after depletion of IRES-vcircRNAs (FIG. 4E). Curiously, 5′ UTR-containing viral RNA abundances were diminished less than core-containing vira RNAs (FIG. 4E, FIG. 6D), possibly due to a higher resistance to degradation of the very structured 5′ UTR.

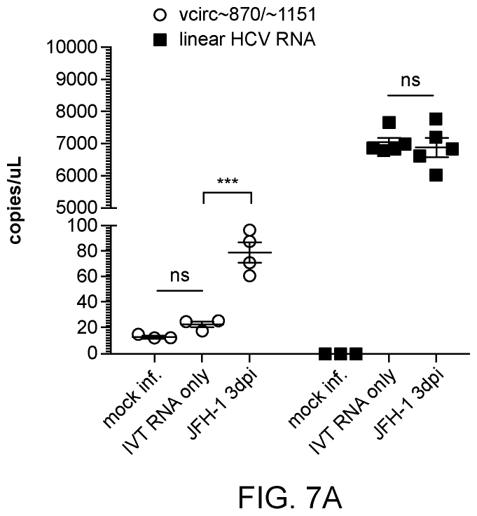

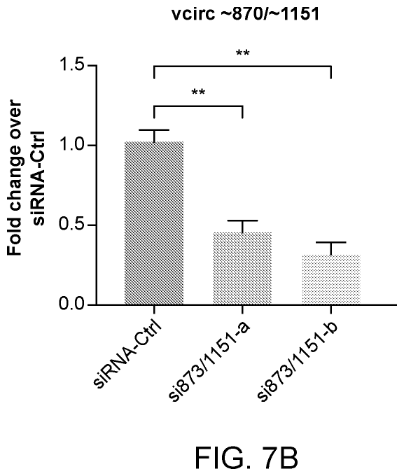

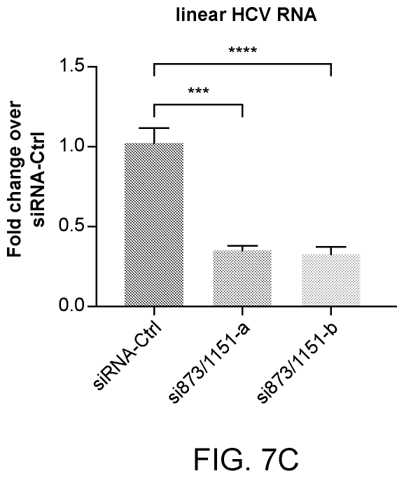

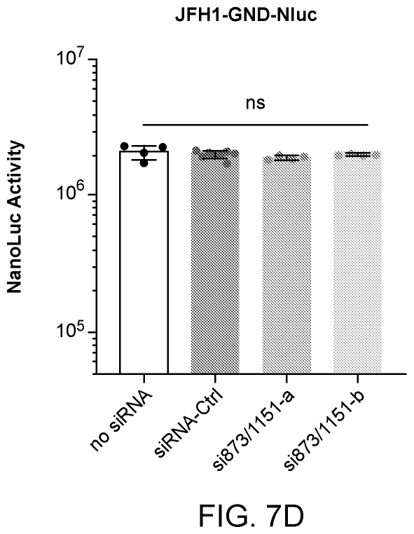

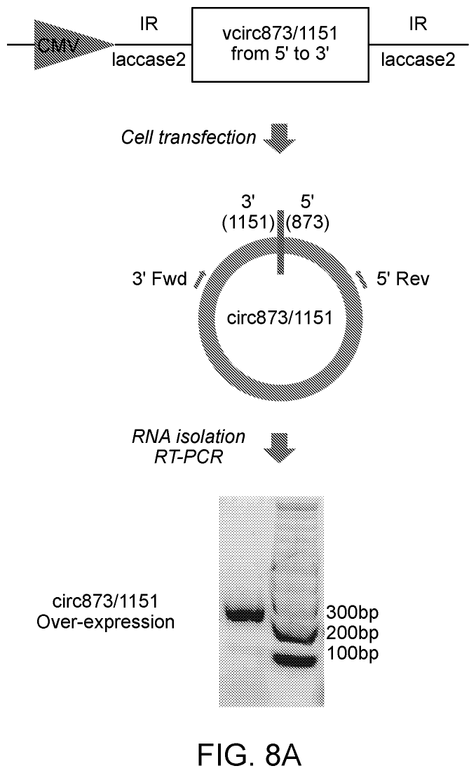

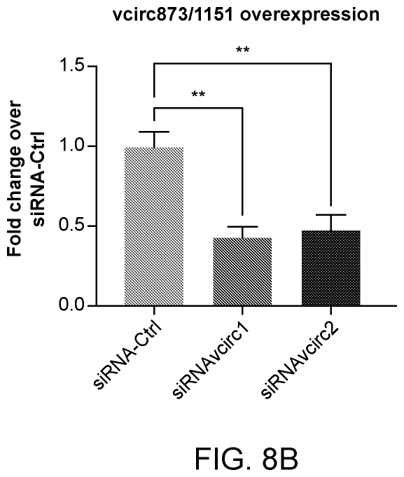

To study functional roles for vcircRNAs in the most abundant ˜870/˜1151 cluster, vcircRNA abundances were first determined by droplet digital PCR. The copy number of these vcircRNAs is 1% of that of linear viral RNAs in infected cells (FIG. 7A). In control experiments using in vitro-transcribed full-length viral RNA, linear HCV RNAs can be easily detected by ddPCR, while only background levels of vcircRNAs could be observed (FIG. 7A), further supporting that vcircRNAs are only produced during viral infection. Next, specific siRNAs that target the junction sequence in the vcircRNA 873/1151 were designed. FIG. 7B shows that two distinct siRNAs significantly depleted vcircRNA cluster˜870/˜1151 not only in HCV-infected cells (FIG. 7B), but also after exogenous overexpression (FIG. 8A, B). The significant reduction in viral RNA abundance that accompanied this siRNA treatment (FIG. 7C) supports the hypothesis that these vcircRNAs have a pro-viral function. Transfection of the two vcircRNA targeting siRNAs into cells expressing the replication-deficient viral genome (FIG. 6) did not alter the amount of luciferase produced (FIG. 7D), arguing that the siRNAs do not display off target effects on linear viral RNA sequences. These results suggest that vcircRNA873/1151 in the most abundant ˜870/˜1151 cluster, together with IRES-containing vcircRNAs, have pro-viral functions in the infectious cycle of HCV.

Sequences
SEQ ID NO: 1
TGAGCACAAATCCTAAACCTCA
SEQ ID NO: 2
TTCCTCACAGGGGAGTGATT
SEQ ID NO: 3
CAAATCCTAAACCTCAAAGAAAAACC
SEQ ID NO: 4
CTCACAGGGGAGTGATTCATGG
SEQ ID NO: 5
CCAGTCTCGCCAAACATGGCTGT
SEQ ID NO: 6
CACCATGTAGCTGCTACTGGTATTCTTCA
SEQ ID NO: 7
GACGCACATCGATATGGTTGTGATGT
SEQ ID NO: 8
GGAACGGTGATGCAGGACAACA
SEQ ID NO: 9
CCCTATATGGGAATGAGGGACT
SEQ ID NO: 10
CGTTAGGGTGTCGATGACTTTAC
SEQ ID NO: 11
CGAGATGTGATGAAGGAGATGG
SEQ ID NO: 12
TTGATGTAATCCAGCAGGTCAG
SEQ ID NO: 13
CTTGGTACCGAGCTCGGATCCACTAGTCCAGTGTGGTGGAAT
SEQ ID NO: 14
TTATTAATTAAGTCGACTGCAGAATTCAGATCC
SEQ ID NO: 15
GTCCTGCAGGGTATGGTGAGCAAGGGCGAGGAGCT
SEQ ID NO: 16
AACGGGCCCTCTAGACTCGAGCGGCCGCCTGAGGTGCCAC
SEQ ID NO: 17
GCAGTCGACTTAATTAATAATAGGGGCGACACTCCGCC
SEQ ID NO: 18
CTCACCATACCCTGCAGGACGTCTTCTGGGCGACGGTTG
SEQ ID NO: 19
CGCCACCUUGCUGGCCCUGUUdTdT
SEQ ID NO: 20
GCCACCUUGCUGGCCCUGUUGdTdT
SEQ ID NO: 21
GAUCAUACGUGCGAUCAGAdTdT
SEQ ID NO: 22
UCUGAUCGCACGUAUGAUCdTdT
SEQ ID NO: 23
AAAAGCAGGCTACTCGAATTCTAATACGACT
SEQ ID NO: 24
AACAGCCGCCAGCCGCTCACGGGAGTGATTCCTGGCGGAGT
SEQ ID NO: 25
AGTTAGCTAATCTTCTTGAACAGCCGCCAGCCGCT
SEQ ID NO: 26
TTCAAGAAGATTAGCTAACTGTGAGGAACTACTGTCTTC
SEQ ID NO: 27
CACGCGATGCCATCGCGGCC
SEQ ID NO: 28
CGCTCACGGGAGTGATTCATGGCGGAGTGTC
SEQ ID NO: 29
GCCGCCAGCCGCTCACGGGAGTGATT
SEQ ID NO: 30
ATCGAAATTAATACGACTCACTATAGGACCTGCCCCTAATAGGGGC
SEQ ID NO: 31
GCCCTCTAGACTCGAACCCAGTCTTCCCGGCAA
SEQ ID NO: 32
TTAACGTCTTCTGGGCGACGGTT
SEQ ID NO: 33
UGGAGUGUGACAAUGGUGUUUGU
SEQ ID NO: 34
ACCUGCCCCUAAUAGGGGCGACACUCCGCCAUGAAUCACUCCCCU
SEQ ID NO: 35
VSGWRLFKKIS
SEQ ID NO: 36
ACCTGCCCCTAATAGGGGCGACACTCCGCCAGGAATCACTCCCGTGAG
CGGCTGGCGGCTGTTCAAGAAGATTAGCTAACT
SEQ ID NO: 37
ACCTGCCCCTAATAGGGGCGACACTCCGCCATGAATCACTCCCGTGAG
CGGCTGGCGGCTGTTCAAGAAGATTAGCTAACT
SEQ ID NO: 38
TGCTGGCCCTGTTGTCCTGCATCACCGTTCCGGTCTCTGCTGCCCAGG
TGAAAAATACCAGTAGCAGCTACATGGTGACCAATGACTGCTCCAATG
ACAGCATCACTTGGCAGCTCGAGGCTGCGGTTCTCCACGTCCCCGGGT
GCGTCCCGTGCGAGAGAGTGGGGAATACGTCACGGTGTTGGGTGCCAG
TCTCGCCAAACATGGCTGTGCGGCAGCTCGGTGCCCTCACGCAGGGTC
TGCGGACGCACATCGATATGGTTGTGATGTCCGCCACCT
SEQ ID NO: 39
TCTGCAGTCGACTTACATGAATCACTCCCCTGTGA
SEQ ID NO: 40
TTGCTCACCATACCCGTTTTTCTTTGAGGTTTAGGATTTG
SEQ ID NO: 41
TCTGCAGTCGACTTAATTAAAGGGGCGACACTCCGCCATG
SEQ ID NO: 42
TTGCTCACCATACCCAGAGGTTTAGGATTTGTGCTCATGGT
SEQ ID NO: 43
TCTGCAGTCGACTTACCGACATGAATCACTCCCCT
SEQ ID NO: 44
TTGCTCACCATACCCTTTCTTTGAGGTTTAGGATTTGTG
SEQ ID NO: 45
TCTGCAGTCGACTTATTGCTGGCCCTGTTGTCC
SEQ ID NO: 46
TTGCTCACCATACCCGGTGGCGGACATCACAACC
SEQ ID NO: 47
GCCACCTTGCTGGCCCTG
SEQ ID NO: 48
CGCCACCTTGCTGGCCCT
SEQ ID NO: 49
GTCCGCCACCTTCATCT
SEQ ID NO: 50
CCGCCACCTTCTATCTT
SEQ ID NO: 51
ACCUGCCCCUAAUAGGGGCGACACUCCGCCAGGAAUCACUCCCCU
SEQ ID NO: 52
GAAGACGTTAATAGGGGC
SEQ ID NO: 53
GAAGACGTTAATAGGGGC
SEQ ID NO: 54
CCTAAACCTCAGGGGCGA
SEQ ID NO: 55
CTAAACCTCAGGGGCGA

1. A method of reducing a proviral effect of a virus-derived circular RNA on a target cell, the method comprising:
(a) identifying a virus-derived circular RNA having a proviral effect, wherein the virus-derived circular RNA is derived from a genome of an RNA virus; and
(b) contacting the target cell with an agent that blocks a proviral function of the virus-derived circular RNA.

2. The method of claim 1, wherein the RNA virus is a cytoplasmic RNA virus selected from the group consisting of a flavivirus, a coronavirus, an enterovirus, a reovirus, a picornavirus, and a togavirus.

3. The method of claim 2, wherein the cytoplasmic RNA virus is a flavivirus and wherein the flavivirus is selected from the group consisting of hepatitis C virus, Zika virus, West Nile virus, Dengue virus, yellow fever virus, and St. Louis encephalitis virus.

4. (canceled)

5. The method of claim 2, wherein the cytoplasmic RNA virus is a coronavirus.

6. (canceled)

7. The method of claim 1, wherein the RNA virus is a nuclear RNA virus selected from the group consisting of orthomyxovirus and retrovirus.

8. (canceled)

9. The method of claim 1, wherein the agent that blocks a proviral function of the virus-derived circular RNA is selected from the group consisting of an siRNA, an shRNA, an RNA-targeting CRISPR-Cas system, and a small molecule.

10. The method of claim 1, wherein the agent that blocks a proviral function of the virus-derived circular RNA targets a back splicing or junction sequence of the virus-derived circular RNA.

11. The method of claim 9, wherein the agent that blocks a proviral function of the virus-derived circular RNA is an siRNA and wherein the siRNA comprises a nucleotide sequence of a junction sequence of the virus-derived circular RNA.

12. The method of claim 9, wherein the agent that blocks a proviral function of the virus-derived circular RNA is an siRNA and wherein the siRNA comprises a nucleotide sequence of a transcription regulatory sequence of the virus-derived circular RNA.

13-15. (canceled)

16. A method of promoting an innate immune response in a subject, the method comprising administering to the subject a virus-derived circular RNA derived from a genome of an RNA virus, wherein the virus-derived circular RNA promotes in the subject an innate immune response to the RNA virus.

17. The method of claim 16, wherein the virus-derived circular RNA is administered to the subject by a nanoparticle containing the virus-derived circular RNA or by a vector that encodes the virus-derived circular RNA.

18. A method of promoting an innate immune response in a subject, the method comprising administering to the subject an agent that blocks a function of a virus-derived circular RNA derived from a genome of an RNA virus and wherein the virus-derived circular RNA reduces in the subject an innate immune response to the RNA virus.

19. The method of claim 18, wherein the agent that blocks a function of the virus-derived circular RNA is selected from the group consisting of an siRNA, an shRNA, a RNA-targeting CRISPR-Cas system, and a small molecule.

20. (canceled)

21. A method of treating an RNA viral infection in a subject, the method comprising:
(a) detecting in a biological sample from the subject a virus-derived circular RNA, wherein the virus-derived circular RNA is derived from a genome of an RNA virus; and
(b) administering to the subject a therapeutically effective amount of an antiviral agent.

22. The method of claim 21, wherein the antiviral agent is an agent that blocks a function of the virus-derived circular RNA and wherein the agent that blocks a function of the virus-derived circular RNA is selected from the group consisting of an siRNA, an shRNA, a RNA-targeting CRISPR-Cas system, and a small molecule.

23. (canceled)

24. A method of treating an RNA viral infection in a subject, comprising:
(a) detecting in a biological sample from the subject a polypeptide translated from a virus-derived circular RNA, wherein the virus-derived circular RNA is derived from a genome of a cytoplasmic RNA virus; and
(b) administering to the subject a therapeutically effective amount of an antiviral agent.

25. The method of claim 24, wherein the antiviral agent is an agent that blocks a function of the virus-derived circular RNA and wherein the agent that blocks a function of the virus-derived circular RNA is selected from the group consisting of an siRNA, an shRNA, a RNA-targeting CRISPR-Cas system, and a small molecule.

26. (canceled)

27. A nanoparticle comprising:
(a) an agent that blocks a function of a virus-derived circular RNA; and
(b) a polymer layer encapsulating the agent.

28. The nanoparticle of claim 27, wherein the agent that blocks a function of a virus-derived circular RNA is selected from the group consisting of an siRNA, an shRNA, a RNA-targeting CRISPR-Cas system, and a small molecule.

29. The nanoparticle of claim 28, wherein the siRNA, shRNA, or RNA-targeting CRISPR-Cas system or small molecule targets a back splicing junction of the virus-derived circular RNA.

30. A vector encoding an siRNA, an shRNA, a RNA-targeting CRISPR-Cas system, or a small molecule that targets a virus-derived circular RNA, wherein the virus-derived circular RNA is derived from a genome of an RNA virus.

31. A nanoparticle comprising:
(a) a vector encoding an siRNA, an shRNA, or a RNA-targeting CRISPR-Cas system that targets a virus-derived circular RNA, wherein the virus-derived circular RNA is derived from a genome of an RNA virus; and
(b) a polymer layer encapsulating the vector.

32. A method of modulating an effect of a virus-derived circular RNA on one or more uninfected bystander cells, the method comprising contacting the one or more uninfected bystander cells with a therapeutically effective amount of an agent that blocks a proviral effect of the virus-derived circular RNA on the one or more uninfected bystander cells or an agent that promotes an antiviral effect of the virus-derived circular RNA in the one or more uninfected bystander cells.
